(19) United States
(12) Patent Application Publication
(10) Pub. No.: US 2026/0138973 A1
(43) Pub. Date: May 21, 2026

(54) SUBSTITUTED PYRIDINES FOR USE IN TREATING OR PREVENTING INFLAMMATORY DISEASES OR DISEASES ASSOCIATED WITH AN UNDESIRABLE IMMUNE RESPONSE

(71) Applicant: SITRYX THERAPEUTICS LIMITED, Oxford (GB)

(72) Inventors: Michael Liam COOKE, Nottingham, Nottinghamshire (GB); Alessandro MAZZACANI, Oxford (GB)

(21) Appl. No.: 19/138,467

(22) PCT Filed: Dec. 15, 2023
(86) PCT No.: PCT/GB2023/053261

§ 371 Date: Jun. 12, 2025

(30) Foreign Application Priority Data
Dec. 15, 2022 (EP) 22213749.9

(51) Int. Cl. C07D 413/04; A61K 31/122; A61K 31/203; A61K 31/397; A61K 31/4035; A61K 31/436; A61K 31/4436; A61K 31/4439; A61K 31/519; A61K 31/52; A61K 31/593; A61K 31/69; A61P 37/06

(52) U.S. Cl. CPC C07D 413/04; A61K 31/122; A61K 31/203; A61K 31/397; A61K 31/4035; A61K 31/436; A61K 31/4436; A61K 31/4439; A61K 31/519; A61K 31/52; A61K 31/593; A61K 31/69; A61P 37/06

(57) ABSTRACT

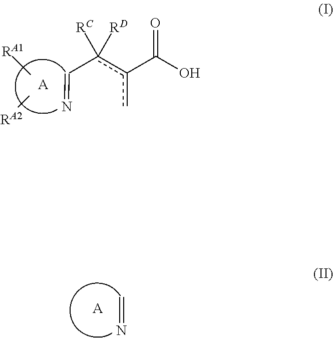

The invention relates to compounds of formula (I) and to their use in treating or preventing an inflammatory disease or a disease associated with an undesirable immune response: (I) wherein, (II), R^{A1}, R^{A2}, R^{C} and R^{D} are as defined herein.

### FIELD OF THE INVENTION

The present invention relates to compounds for use in treating or preventing inflammatory diseases or diseases associated with an undesirable immune response, and to related compositions, methods, uses and intermediate compounds.

### BACKGROUND OF THE INVENTION

Chronic inflammatory diseases such as rheumatoid arthritis, systemic lupus erythematosus (SLE), multiple sclerosis, psoriasis, Crohn's disease, ulcerative colitis, uveitis and chronic obstructive pulmonary disease (COPD) represent a significant burden to society because of life-long debilitating illness, increased mortality and high costs for therapy and care (Straub R. H. and Schradin C., 2016). Non-steroidal anti-inflammatory drugs (NSAIDs) are the most widespread medicines employed for treating inflammatory disorders, but these agents do not prevent the progression of the inflammation and only treat the accompanying symptoms. Glucocorticoids are powerful anti-inflammatory agents, making them emergency treatments for acute inflammatory flares, but given longer term these medicines give rise to a plethora of unwanted side-effects and may also be subject to resistance (Straub R. H. and Cutolo M., 2016). Thus, considerable unmet medical need still exists for the treatment of inflammatory disorders and extensive efforts to discover new medicines to alleviate the burden of these diseases is ongoing (Hanke T. et al., 2016).

Dimethyl fumarate (DMF), a diester of the citric acid cycle (CAC) intermediate fumaric acid, is utilised as an oral therapy for treating psoriasis (Bruck J. et al., 2018) and multiple sclerosis (Mills E. A. et al., 2018). Importantly, following oral administration, none of this agent is detected in plasma (Dibbert S. et al., 2013), the only drug-related compounds observed being the hydrolysis product monomethyl fumarate (MMF) and glutathione (GSH) conjugates of both the parent (DMF) and metabolite (MMF). DMF's mechanism of action is complex and controversial. This compound's efficacy has been attributed to a multiplicity of different phenomena involving covalent modification of proteins and the conversion of “prodrug” DMF to MMF. In particular, the following pathways have been highlighted as being of relevance to DMF's anti-inflammatory effects: 1) activation of the anti-oxidant, anti-inflammatory, nuclear factor (erythroid-derived 2)-like 2 (NRF2) pathway as a consequence of reaction of the electrophilic α,β-unsaturated ester moiety with nucleophilic cysteine residues on kelch-like ECH-associated protein 1 (KEAP1) (Brennan M. S. et al., 2015); 2) induction of activating transcription factor 3 (ATF3), leading to suppression of pro-inflammatory cytokines interleukin (IL)-6 and IL-8 (Müller S. et al., 2017); 3) inactivation of the glycolytic enzyme glyceraldehyde 3-phosphate dehydrogenase (GAPDH) through succination of its catalytic cysteine residue with a Michael accepting unsaturated ester (Kornberg M. D. et al., 2018; Angiari S. and O'Neill L. A., 2018); 4) inhibition of nuclear factor-kappaB (NF-κB)-driven cytokine production (Gillard G. O. et al., 2015); 5) prevention of the association of PKCθ with the costimulatory receptor CD28 to reduce the production of IL-2 and block T-cell activation (Blewett M. M. et al., 2016); 6) reaction of the electrophilic α, β-unsaturated ester with the nucleophilic thiol group of anti-oxidant GSH, impacting cellular responses to oxidative stress (Lehmann J. C. U. et al., 2007); 7) agonism of the hydroxycarboxylic acid receptor 2 (HCA2) by the MMF generated in vivo through DMF hydrolysis (von Glehn F. et al., 2018); 8) allosteric covalent inhibition of the p90 ribosomal S6 kinases (Andersen J. L. et al., 2018); 9) inhibition of the expression and function of hypoxia-inducible factor-1α (HIF-1α) and its target genes, such as IL-8 (Zhao G. et al., 2014); and 10) inhibition of Toll-like receptor (TLR)-induced M1 and K63 ubiquitin chain formation (McGuire V. A. et al., 2016). In general, with the exception of HCA2 agonism (Tang H. et al., 2008), membrane permeable diester DMF tends to exhibit much more profound biological effects in cells compared to its monoester counterpart MMF. However, the lack of systemic exposure of DMF in vivo has led some researchers to assert that MMF is, in fact, the principal active component following oral DMF administration (Mrowietz U. et al., 2018). As such, it is evident that some of the profound biology exerted by DMF in cells is lost because of hydrolysis in vivo to MMF.

Recently, it has been discovered that, during inflammatory macrophage activation, the CAC becomes anaplerotic and is diverted such that the unsaturated diacid itaconic acid, “itaconate”, is generated (Murphy M. P. and O'Neill L. A. J., 2018; O'Neill L. A. J. and Artyomov M. N., 2019; Yu X.-H. et al., 2019). Instead of being hydrated to isocitrate by aconitate hydratase, the CAC intermediate aconitate is decarboxylated by the protein product of immune-responsive gene 1 (IRG1), one of the most highly upregulated genes in macrophages under proinflammatory conditions, subsequently named aconitate decarboxylase 1, to produce itaconic acid (Michelucci A. et al., 2013). This unsaturated diacid is an inhibitor of the bacterial enzyme isocitrate lyase and, as such, it exerts anti-bacterial activity. In addition, itaconic acid has been shown to inhibit the CAC enzyme succinate dehydrogenase (SDH) (Ackermann et al., 1949), leading accordingly to succinate accumulation (Cordes T. et al., 2016). By inhibiting SDH, an enzyme critical for the inflammatory response (E. L. Mills et al., 2016), itaconate ameliorates inflammation in vitro and in vivo during macrophage activation and ischemia-reperfusion injury (Lampropoulou V. et al., 2016).

Like fumaric acid, itaconic acid is an α,β-unsaturated carboxylic acid. As such, it is a Michael acceptor which induces a global electrophilic stress response. In this regard, the itaconic acid diester dimethyl itaconate (DMI), like DMF, produces an anti-inflammatory response, reducing the expression levels of pro-inflammatory cytokines IL-1β, IL-6, IL-12 and IL-18 in lipopolysaccharide (LPS)-stimulated bone marrow-derived macrophages (WO2017/142855A1, incorporated herein by reference). This response appears to be mediated, in part, by NRF2 activation, via alkylation of KEAP1 cysteine residues by the electrophilic α,β-unsaturated ester moiety (Mills E. L. et al., 2018), which enhances the expression of downstream genes with anti-oxidant and anti-inflammatory capacities. Nevertheless, not all of the pronounced immunoregulatory effects engendered by DMI can be attributed to NRF2 activation. In particular, the modulation of IκBζ by DMI is independent of NRF2 and is mediated via upregulation of ATF3, a global negative regulator of immune activation that downregulates various cytokines, such as IL-6 (Bambouskova M. et al., 2018). Moreover, by inhibiting IκBζ protein production, DMI ameliorates IL-17-mediated pathologies, highlighting the therapeutic potential of this regulatory pathway (WO2019/036509A1, incorporated herein by reference). Further highlighting its pharmacologic potential, DMI has recently been reported to 1) demonstrate a protective effect on cerebral ischemia/reperfusion injury, thereby offering potential for the treatment of ischemic stroke (Zhang D. et al., 2019); 2) provide protection from the cardiotoxic effects of doxorubicin (Shan Q. et al., 2019); 3) protect against lippolysacchride-induced mastitis in mice by activating MAPKs and NRF2 while inhibiting NF-κB signaling pathways (Zhao C. et al., 2019). Furthermore, DMI is said to have utility in preventing and treating ulcerative colitis and canceration thereof (CN110731955, Sun Yat-sen University Cancer Center); and has been reported to protect against fungal keratitis by activating the NRF2/HO-1 signalling pathway (Gu L. et al., 2020). Nevertheless, it should be noted that DMI is not metabolised to itaconic acid intracellularly (ElAzzouny M. et al., 2017). Other α,β-unsaturated esters and acids exhibit IL-1β-lowering effects in macrophages by inhibiting the NLRP3 inflammasome (Cocco M. et al., 2017 and 2014), and have been demonstrated to inhibit the TLR4 pathway, leading ultimately to suppression of LPS-induced stimulation of NF-κB, tumour necrosis factor (TNF)-α, IL-1β and nitric oxide release (Zhang S. et al., 2012). WO2014/152263A1 (Karyopharm Therapeutics, Inc.) describes α,β-unsaturated esters which are said to be chromosomal region maintenance 1 (CRM1) inhibitors. CRM-1 plays a role in exporting several key proteins that are involved in many inflammatory processes.

Other itaconic acid derivatives have been demonstrated to elicit anti-inflammatory effects (Bagavant G. et al., 1994). A notable example is 4-octyl itaconic acid (401), an itaconate derivative with improved cellular uptake. Since the α,β-unsaturated carboxylic acid is not esterified in 401, this electrophile exhibits low reactivity with biological thiols (Schmidt T. J. et al., 2007), much like the situation encountered with itaconic acid itself. As a result of its low reactivity/electrophilicity, the NRF2-activating effects of 401 are not attenuated by GSH, in contrast to the findings with the much more reactive DMI. In this latter case, the α,β-unsaturated carboxylic acid is esterified and, as a consequence, the IL-6-lowering and NRF2-activating effects of DMI are reversed by the thiols N-acetylcysteine and GSH, respectively. Through the reaction with KEAP1 and the resulting NRF2 activation, as well as GAPDH inhibition (Liao S.-T. et al., 2019), 401 has been demonstrated to produce a wide range of interesting biological effects, including: 1) protection of neuronal cells from hydrogen peroxide (Liu H. et al., 2018); 2) inhibition of proinflammatory cytokine production in peripheral blood mononuclear cells of SLE patients (Tang C. et al., 2018); 3) protection of human umbilical vein endothelial cells from high glucose (Tang C. et al., 2019); 4) inhibition of osteoclastogenesis by suppressing the E3 ubiquitin ligase Hrd1 and activating NRF2 signaling (Sun X. et al., 2019); 5) induction of repression of STING by NRF2 and type I IFN production in cells from patients with STING-dependent interferonopathies (Olagnier D. et al., 2018); 6) protection against renal fibrosis via inhibiting the TGF-beta/Smad pathway, autophagy and reducing generation of reactive oxygen species (Tian F. et al., 2020); 7) reduction of brain viral burden in mice intracranially injected with Zika virus (Daniels B. P. et al. 2019); and 8) protection against liver ischemia-reperfusion injury (Yi F. et al. 2020). Furthermore, itaconate has been reported to modulate tricarboxylic acid and redox metabolism to mitigate reperfusion injury (Cordes T. et al., 2020). In addition, raised plasma itaconate levels demonstrate a clear correlation with reduction in rheumatoid arthritis disease activity scores following commencement of therapy with conventional disease modifying anti-rheumatic drug (cDMARD) therapy (Daly R. et al., 2019).

Artyomov et al. (WO2017/142855; WO2019/036509) disclose the use of itaconate, malonate or a derivative thereof as an immunomodulatory agent.

WO2020/222011, WO2020/222010, WO2021/130492, WO2022/029438, WO2022/038365, WO2022/090723, WO2022/090714, WO2022/090724, WO2022/229617, WO2022/269251, WO2023/017269, PCT/GB2023/051633 and PCT/GB2023/052791 (Sitryx Therapeutics Limited) all disclose certain itaconate derivatives.

In spite of the above findings, there remains a need to identify and develop new α,β-unsaturated carboxyl compounds such as itaconate and acrylate derivatives possessing enhanced properties compared to currently marketed anti-inflammatory agents, such as DMF. The present inventors have now discovered, surprisingly, that certain α,β-unsaturated methacrylic acids possessing heteroaryl groups are effective at reducing cytokine release, activating NRF2 in cells and/or have improved metabolic stability. Such compounds are therefore expected to possess excellent anti-inflammatory properties and these properties make them potentially more effective than 4-octyl itaconate in particular.

### SUMMARY OF THE INVENTION

The present invention provides a compound of formula (I):

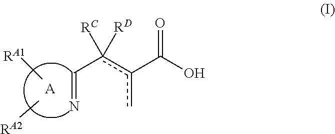

wherein,

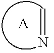

represents a 5 membered heteroaryl ring, which in addition to the C═N shown contains one or more further heteroatoms independently selected from N, O and S;
or

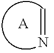

represents a 6 membered heteroaryl ring, which in addition to the C═N shown optionally contains one or more further N atoms;
R^{A1} is —(CH₂)₀₋₆-heteroaryl or O-heteroaryl;
   wherein R^{A1} is optionally substituted by one or more substituents selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more groups selected from halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms;
   or R^{A1} is optionally substituted by one phenyl ring which is optionally substituted by C₁₋₂ haloalkyl, C₁₋₂ haloalkoxy or one or more halo atoms;
wherein G¹ is C₁₋₆ alkyl, C₃₋₇ cycloalkyl, C₁₋₆ haloalkyl, or (CH₂)₀₋₁phenyl, wherein G¹ is optionally substituted by one or more substituents selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR¹R², OG² and S(O)₀₋₂G²;
   wherein G² is C₁₋₆ alkyl, C₃₋₇ cycloalkyl, C₁₋₆ haloalkyl, or phenyl which is optionally substituted by one or more substituents selected from the group consisting of halo, C₁₋₂ alkyl, C₁₋₂ haloalkyl, hydroxy, cyano, nitro, C₁₋₂ alkoxy and C₁₋₂ haloalkoxy; and wherein R¹ and R² are independently H or C₁₋₂ alkyl or, taken together, R¹ and R² may combine to form a 5-7-membered heterocyclic ring;
or R^{A2} is absent; and
R^{C} and R^{D} are each independently H, C₁₋₂ alkyl, hydroxy, fluoro or C₁₋₂ alkoxy; or R^{C} and R^{D} may join to form a C₃₋₅ cycloalkyl ring;
wherein

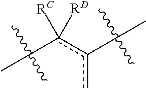

in the compound of formula (I) represents:

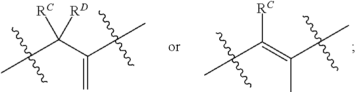

and wherein
the total number of carbon atoms in groups R^{A1} and R^{A2} taken together including their optional substituents is 6-14;
or a pharmaceutically acceptable salt and/or solvate thereof.

The present invention provides a pharmaceutical composition comprising a compound of formula (I) or a pharmaceutically acceptable salt and/or solvate thereof.

The present invention provides a compound of formula (I) or a pharmaceutically acceptable salt and/or solvate thereof for use as a medicament.

The present invention provides a compound of formula (I) or a pharmaceutically acceptable salt and/or solvate thereof for use in treating or preventing an inflammatory disease or a disease associated with an undesirable immune response.

The present invention provides the use of a compound of formula (I) or a pharmaceutically acceptable salt and/or solvate thereof in the manufacture of a medicament for treating or preventing an inflammatory disease or a disease associated with an immune response.

The present invention provides a method of treating or preventing an inflammatory disease or a disease associated with an undesirable immune response, which comprises administering a compound of formula (I) or a pharmaceutically acceptable salt and/or solvate thereof.

Also provided are intermediate compounds of use in the preparation of compounds of formula (I).

### DETAILED DESCRIPTION OF THE INVENTION

### Compounds of Formula (I)

Embodiments and preferences set out herein with respect to the compound of formula (I) apply equally to the pharmaceutical composition, compound for use, use and method aspects of the invention.

The term “C₁₋₁₀ alkyl” refers to a straight or branched fully saturated hydrocarbon group having from 1 to 10 carbon atoms. The term encompasses methyl, ethyl, n-propyl, isopropyl, n-butyl, isobutyl, sec-butyl, tert-butyl, n-pentyl, n-heptyl, n-hexyl and n-octyl. Other branched variants such as heptyl-CH(CH₃)- and hexyl-CH(CH₃)— are also included. Other alkyl groups, for example C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₂₋₁₀ alkyl, C₂₋₉ alkyl, C₂₋₈ alkyl, C₂₋₇ alkyl, C₂₋₆ alkyl, C₂₋₅ alkyl, C₂₋₄ alkyl, C₂₋₃ alkyl, C₃₋₁₀ alkyl, C₃₋₉ alkyl, C₃₋₈ alkyl, C₃₋₇ alkyl, C₃₋₆ alkyl, C₃₋₅ alkyl, C₃₋₄ alkyl, C₄₋₁₀ alkyl, C₄₋₉ alkyl, C₄₋₈ alkyl, C₄₋₇ alkyl, C₄₋₆ alkyl, C₄₋₅ alkyl, C₅₋₁₀ alkyl, C₅₋₉ alkyl, C₅₋₈ alkyl, C₅₋₇ alkyl, C₅₋₆ alkyl, C₆₋₁₀ alkyl, C₆₋₉ alkyl, C₆₋₈ alkyl, C₇₋₁₀ alkyl, C₇₋₉ alkyl, C₇₋₈ alkyl, C₈₋₁₀ alkyl, C₈₋₉ alkyl and C₉₋₁₀ alkyl are as defined above but contain different numbers of carbon atoms. The term “C₁₋₁₀ alkyl” also encompasses “C₁₋₁₀ alkylene” which is a bifunctional straight or branched fully saturated hydrocarbon group having the stated number of carbon atoms. Example “alkylene” groups include methylene, ethylene, n-propylene, n-butylene, n-pentylene, n-hexylene, n-heptylene, n-octylene, and stereoisomers thereof such as 2-propylene, 2-butylene, 2-pentylene, 3-pentylene, 2-hexylene, 3-hexylene, 2-heptylene, 3-heptylene, 4-heptylene, 2-octylene, 3-octylene and 4-octylene.

The term “C₂₋₁₀ alkenyl” refers to a straight or branched hydrocarbon group having from 2 to 10 carbon atoms and at least one carbon-carbon double bond. The term encompasses, CH═CH₂, CH₂CH═CH₂, CH═CHCH₃, CH₂CH₂CH═CH₂, CH═CHCH₂CH₃, CH₂CH═CHCH₃, CH₂CH₂CH₂CH═CH₂, CH═CHCH₂CH₂CH₃, CH₂CH═CHCH₂CH₃, CH₂CH₂CH═CHCH₃, CH═CHCH═CHCH₃ and CH₂CH═CHCH═CH₂. Branched variants such as CH(CH₃)CH═CH₂ and CH═C(CH₃)CH₂ are also included. Other alkenyl groups, for example C₂₋₉ alkenyl, C₂₋₈ alkenyl, C₂₋₇ alkenyl, C₂₋₆ alkenyl, C₂₋₅ alkenyl, C₂₋₄ alkenyl, C₂₋₃ alkenyl, C₃₋₁₀ alkenyl, C₃₋₉ alkenyl, C₃₋₈ alkenyl, C₃₋₇ alkenyl, C₃₋₆ alkenyl, C₃₋₅ alkenyl, C₃₋₄ alkenyl, C₄₋₁₀ alkenyl, C₄₋₉ alkenyl, C₄₋₈ alkenyl, C₄₋₇ alkenyl, C₄₋₆ alkenyl, C₄₋₅ alkenyl, C₅₋₁₀ alkenyl, C₅₋₉ alkenyl, C₅₋₈ alkenyl, C₅₋₇ alkenyl, C₅₋₆ alkenyl, C₆₋₁₀ alkenyl, C₆₋₉ alkenyl, C₆₋₈ alkenyl, C₇₋₁₀ alkenyl, C₇₋₉ alkenyl, C₇₋₈ alkenyl, C₈₋₁₀ alkenyl, C₈₋₉ alkenyl and C₉₋₁₀ alkenyl are as defined above but contain different numbers of carbon atoms.

The term “C₂₋₁₀ alkynyl” refers to a straight or branched hydrocarbon group having from 2 to 10 carbon atoms and at least one carbon-carbon triple bond. The term encompasses, CECH, CH₂C≡CH, C≡C-CH₃, CH₂CH₂C≡CH, C≡CCH₂CH₃, CH₂C≡CCH₃, CH₂CH₂CH₂C≡CH, C≡CCH₂CH₂CH₃, CH₂C≡CCH₂CH₃, CH₂CH₂C≡CCH₃, C≡CC≡CCH₃ and CH₂C≡CC≡CH. Branched variants such as CH(CH₃)C≡CH are also included. Other alkynyl groups, for example C₂₋₉ alkynyl, C₂₋₈ alkynyl, C₂₋₇ alkynyl, C₂₋₆ alkynyl, C₂₋₅ alkynyl, C₂₋₄ alkynyl, C₂₋₃ alkynyl, C₃₋₁₀ alkynyl, C₃₋₉ alkynyl, C₃₋₈ alkynyl, C₃₋₇ alkynyl, C₃₋₆ alkynyl, C₃₋₅ alkynyl, C₃₋₄ alkynyl, C₄₋₁₀ alkynyl, C₄₋₉ alkynyl, C₄₋₈ alkynyl, C₄₋₇ alkynyl, C₄₋₆ alkynyl, C₄₋₅ alkynyl, C₅₋₁₀ alkynyl, C₅₋₉ alkynyl, C₅₋₈ alkynyl, C₅₋₇ alkynyl, C₅₋₆ alkynyl, C₆₋₁₀ alkynyl, C₆₋₉ alkynyl, C₆₋₈ alkynyl, C₇₋₁₀ alkynyl, C₇₋₉ alkynyl, C₇₋₈ alkynyl, C₈₋₁₀ alkynyl, C₈₋₉ alkynyl and C₉₋₁₀ alkynyl are as defined above but contain different numbers of carbon atoms.

The term “C₃₋₁₀ cycloalkyl” refers to a fully saturated cyclic hydrocarbon group having from 3 to 10 carbon atoms. The term encompasses cyclopropyl, cyclobutyl, cyclopentyl, cyclohexyl, cycloheptyl, cyclooctyl, cyclononyl and cyclodecyl as well as bridged systems such as bicyclo[1.1.1]pentyl, bicyclo[2.2.1]heptyl, bicyclo[2.2.2]octyl and adamantyl. Other cycloalkyl groups, for example C₃₋₉ cycloalkyl, C₃₋₈ cycloalkyl, C₃₋₇ cycloalkyl, C₃₋₆ cycloalkyl, C₃₋₅ cycloalkyl, C₃₋₄ cycloalkyl, C₄₋₁₀ cycloalkyl, C₄₋₉ cycloalkyl, C₄₋₈ cycloalkyl, C₄₋₇ cycloalkyl, C₄₋₆ cycloalkyl, C₄₋₅ cycloalkyl, C₅₋₁₀ cycloalkyl, C₅₋₉ cycloalkyl, C₅₋₈ cycloalkyl, C₅₋₇ cycloalkyl, C₅₋₆ cycloalkyl, C₆₋₁₀ cycloalkyl, C₆₋₉ cycloalkyl, C₆₋₈ cycloalkyl, C₆₋₇ cycloalkyl, C₇₋₁₀ cycloalkyl, C₇₋₉ cycloalkyl, C₇₋₈ cycloalkyl, C₈₋₁₀ cycloalkyl, C₈₋₉ cycloalkyl and C₉₋₁₀ cycloalkyl are as defined above but contain different numbers of carbon atoms.

The term “C₅₋₁₀ spirocycloalkyl” refers to a bicyclic cycloalkyl group wherein the two rings are connected through just one atom. The rings can be different or identical. The term encompasses spiro[3.3]heptyl. Other spirocycloalkyl groups, for example C₅₋₉ spirocycloalkyl, C₅₋₈ spirocycloalkyl and C₅₋₇ spirocycloalkyl are as defined above but contain different numbers of carbon atoms.

The term “5-7 membered heterocyclic ring” refers to a non-aromatic cyclic group having 5 to 7 ring atoms and wherein at least one of the ring atoms is a heteroatom selected from N, O, S and B. The term “heterocyclic ring” is interchangeable with “heterocyclyl”. The term encompasses pyrrolidinyl, tetrahydrofuranyl, tetrahydrothienyl, tetrahydropyranyl, piperidinyl, piperazinyl, morpholinyl, thiomorpholinyl and homomorpholinyl. 5-7 membered heterocyclyl groups can typically be substituted by one or more (e.g. one or two) oxo groups. Suitably, thietanyl is substituted by one or two oxo groups. Bicyclic heterocyclic compounds are also encompassed, such as the following:

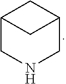

The term “aryl” refers to a cyclic group with aromatic character having from 6 to 10 ring carbon atoms and containing one or two rings. Where an aryl group contains more than one ring, both rings must be aromatic in character. Suitably “aryl” encompasses only phenyl and naphthyl. Most suitably, “aryl” is phenyl.

The term “heteroaryl” such as “5- or 6-membered heteroaryl” refers to a cyclic group with aromatic character containing the indicated number of atoms (e.g. 5 or 6) wherein at least one of the atoms in the cyclic group is a heteroatom independently selected from N, O and S. The term encompasses pyrrolyl, furanyl, thienyl, imidazolyl, pyrazolyl, thiazolyl, oxadiazolyl, thiadiazolyl, triazolyl, oxazolyl, tetrazolyl, pyridyl, pyrimidinyl, pyradizinyl and pyrazinyl.

The term “hydroxy” (which may also be referred to as “hydroxyl”) refers to an —OH group.

The term “halo” as used herein, refers to fluorine, chlorine, bromine or iodine. Particular examples of halo are fluorine and chlorine, especially fluorine.

The term “C₁₋₆ haloalkyl” refers to a C₁₋₆ alkyl group (e.g. a C₁ alkyl group i.e. methyl) as defined above, which is substituted by one or more (e.g., one, two or three) halo atoms. Examples include trifluoromethyl, difluoromethyl, 2,2,2-trifluoroethyl and 1, 1-difluoroethyl.

The term “C₁₋₂ alkoxy” refers to a C₁₋₂ alkyl group (e.g. a C₁ alkyl group i.e. methyl) as defined above, singularly bonded to oxygen. The term encompasses methoxy and ethoxy.

The term “C₁₋₂ haloalkoxy” refers to a C₁₋₂ alkoxy as defined above, which is substituted by one or more (e.g., one, two or three) halo atoms. An example includes trifluoromethoxy.

Where substituents are indicated as being optionally substituted in formula (I) in the embodiments and preferences set out below, said substituents are optionally substituted as specified in the given formula unless stated otherwise, even if the possible substitution is not explicitly listed in the embodiment. Suitably, the optional substituent may be attached to an available carbon atom, which means a carbon atom which is attached to a hydrogen atom i.e. a C—H group. The optional substituent replaces the hydrogen atom attached to the carbon atom.

The group

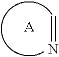

may also be written as

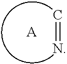

In one embodiment,

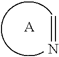

represents a 5 membered heteroaryl ring, which in addition to the C═N shown contains one or more (e.g., one or two) further heteroatoms independently selected from N, O and S.

In one embodiment,

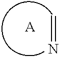

represents a 5 membered heteroaryl ring selected from the group consisting of imidazole, pyrazole, oxazole, isoxazole, thiazole, isothiazole, 1,2,3-triazole, 1,2,4-triazole, 1,2,4-oxadiazole, 1,2,5-oxadiazole, 1,3,4-oxadiazole, 1,2,4-thiadiazole, 1,2,5-thiadiazole, 1,3,4-thiadiazole and tetrazole.

When

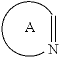

represents imidazole, it is intended to represent

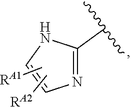

in formula (I). For the avoidance of doubt, substituent R^{A1} and/or R^{A2} (if present) can be bound to a carbon or nitrogen atom of the imidazole moiety.

When

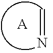

represents pyrazole, it is intended to represent

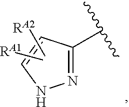

in formula (I). For the avoidance of doubt, substituent R^{A1} and/or R^{A2} (if present) can be bound to a carbon or nitrogen atom of the pyrazole moiety.

When

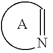

represents oxazole, it is intended to represent in

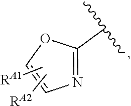

formula (I).

When

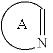

represents isoxazole, it is intended to represent

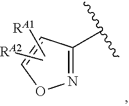

in formula (I).

When

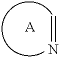

represents thiazole, it is intended to represent

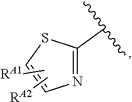

in formula (I).

When

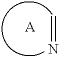

represents isothiazole, it is intended to represent

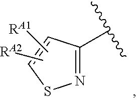

in formula (I).

When

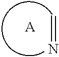

represents 1,2,3-triazole, it is intended to represent

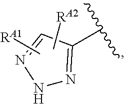

in formula (I). For the avoidance of doubt, substituent R^{A1} and/or R^{A2} (if present) can be bound to a carbon or nitrogen atom of the 1,2,3-triazole moiety.

When

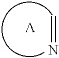

represents 1,2,4-triazole, it is intended to represent

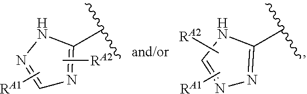

in formula (I). For the avoidance of doubt, substituent R^{A1} and/or R^{A2} (if present) can be bound to a carbon or nitrogen atom of the 1,2,4-triazole moiety.

When

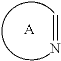

represents 1,2,4-oxadiazole, it is intended to represent

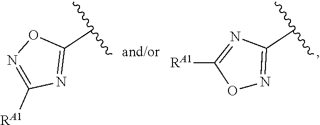

in formula (I).

When

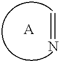

represents 1,2,5-oxadiazole, it is intended to represent

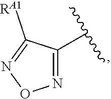

in formula (I).

When

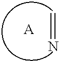

represents 1,3,4-oxadiazole, it is intended to represent

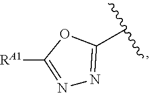

in formula (I).

When

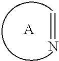

represents 1,2,4-thiadiazole, it is intended to represent

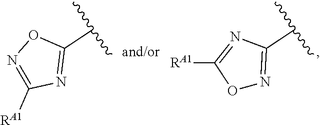

in formula (I).

When

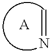

represents 1,2,5-thiadiazole, it is intended to represent

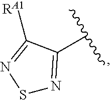

in formula (I).

When

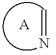

represents 1,3,4-thiadiazole, it is intended to represent

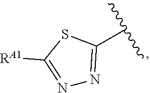

in formula (I).

When

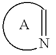

represents tetrazole, it is intended to represent

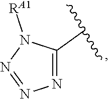

in formula (I).

In one embodiment,

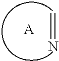

represents an oxadiazole, in particular 1,2,4-oxadiazole. Suitably, the 1,2,4-oxadiazole is

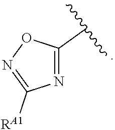

In one embodiment,

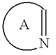

represents 1,3,4-oxadiazole.

In one embodiment,

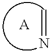

represents a 6 membered heteroaryl ring, which in addition to the C═N shown optionally contains one or more (e.g., one or two) further N atoms.

In one embodiment,

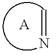

represents a 6 membered heteroaryl ring selected from the group consisting of pyridine, pyridazine, pyrimidine, pyrazine and triazine.

When

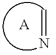

represents pyridine, it is intended to represent

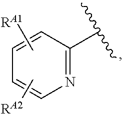

in formula (I).

When

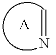

represents pyridazine, it is intended to represent

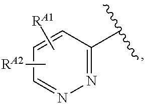

in formula (I).

When

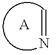

represents pyrimidine, it is intended to represent

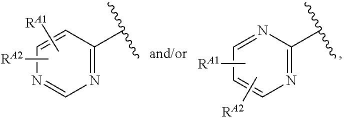

in formula (I).

When

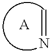

represents pyrazine, it is intended to represent

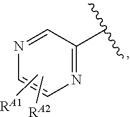

in formula (I).

When

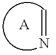

represents triazine, it is intended to represent

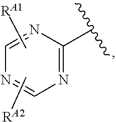

in formula (I).

In the representations above, where a substituent is not indicated as being bound to a carbon atom or nitrogen atom and is instead shown as intersecting a double or single bond of a heteroaryl compound, this indicates that the point of attachment is undefined, and may be any attachment point which is chemically feasible. Furthermore, each of the above mentioned heteroaryl groups is shown as a single tautomer. The skilled person recognises that although a single tautomer is shown, the compound may exist as a mixture of tautomeric forms. Thus, the invention extends to all tautomeric forms of the compounds of formula (I).

In one embodiment, R^{A1} is —(CH₂)₀₋₆-heteroaryl such as —(CH₂)₀-heteroaryl. In another embodiment, R^{A1} is O-heteroaryl. Suitably, the heteroaryl group is 2-pyridyl or 3-pyridyl e.g. 2-pyridyl.

In one embodiment, R^{A1} is not substituted.

In one embodiment, R^{A1} is substituted by one substituent. In another embodiment, R^{A1} is substituted by two substituents. In another embodiment, R^{A1} is substituted by three substituents. Suitably, R^{A1} is substituted by two substituents.

In one embodiment, R^{A1} is substituted by halo, e.g., fluoro, chloro or bromo e.g., chloro. In a second embodiment, R^{A1} is substituted by C₁₋₆ alkyl, e.g., methyl. In a third embodiment, R^{A1} is substituted by C₁₋₆ haloalkyl e.g., CF₃. In a fourth embodiment, R^{A1} is substituted by hydroxy. In a fifth embodiment, R^{A1} is substituted by cyano. In a sixth embodiment, R^{A1} is substituted by OG¹. In a seventh embodiment, R^{A1} is substituted by S(O)₀₋₂G¹. In an eighth embodiment, R^{A1} is substituted by SF₅. In a nineth embodiment, R^{A1} is substituted by (CH₂)₀₋₃C₃₋₇ cycloalkyl wherein said C₃₋₇ cycloalkyl is optionally substituted by one or more groups selected from halo, C₁₋₃ alkyl and C₁₋₃ haloalkyl. In a tenth embodiment, R^{A1} is substituted by 5-7-membered heterocyclyl such as pyrrolidinyl wherein said 5-7-membered heterocyclyl is optionally substituted by one or more groups selected from halo, C₁₋₃ alkyl and C₁₋₃ haloalkyl. In an eleventh embodiment, R^{A1} is substituted by two alkyl groups such as C₁₋₆ alkyl for example C₁₋₂ alkyl wherein the two alkyl groups are attached to the same carbon atom and are optionally joined to form a C₃₋₇ cycloalkyl ring. In a twelfth embodiment, R^{A1} is substituted by one phenyl ring which is optionally substituted by C₁₋₂ haloalkyl, C₁₋₂ haloalkoxy or one or more halo atoms.

When R^{A1} is optionally substituted by C₁₋₆ alkyl and two alkyl groups which are attached to the same carbon atom are optionally joined to form a C₃₋₇ cycloalkyl ring, groups of the following structure form:

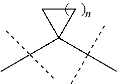

wherein n is an integer selected from 1, 2, 3, 4 and 5. Suitably n is 3.

Suitably, R^{A1} is substituted by one halo group e.g. chloro and one OG¹ wherein G¹ is defined below.

In one embodiment, G¹ is C₁₋₆ alkyl e.g. n-butyl. In a second embodiment, G¹ is C₃₋₇ cycloalkyl, e.g., cyclopropyl. In a third embodiment, G¹ is C₁₋₆ haloalkyl, such as CF₃. In a fourth embodiment, G¹ is (CH₂)₀₋₁phenyl which is optionally substituted by one or more (such as one, two or three, e.g. one) substituents selected from the group consisting of halo, C₁₋₂ alkyl, C₁₋₂ haloalkyl, hydroxy, cyano, nitro, C₁₋₂ alkoxy and C₁₋₂ haloalkoxy. In another embodiment, G¹ is phenyl which is optionally substituted by one or more substituents selected from the group consisting of halo, C₁₋₂ alkyl, C₁₋₂ haloalkyl, hydroxy, cyano, nitro, C₁₋₂ alkoxy and C₁₋₂ haloalkoxy. In another embodiment, G¹ is CH₂-phenyl which is optionally substituted (e.g. the phenyl is substituted) by one or more substituents selected from the group consisting of halo, C₁₋₂ alkyl, C₁₋₂ haloalkyl, hydroxy, cyano, nitro, C₁₋₂ alkoxy and C₁₋₂ haloalkoxy.

Suitably, G¹ is phenyl substituted by halo e.g. fluoro.

In one embodiment, R^{A2} is absent.

In one embodiment, R^{C} is H. In a second embodiment, R^{C} is C₁₋₂ alkyl e.g. methyl. In a third embodiment, R^{C} is hydroxy. In a fourth embodiment, R^{C} is fluoro. In a fifth embodiment, R^{C} is C₁₋₂ alkoxy e.g. OMe.

In one embodiment, R^{D} is H. In a second embodiment, R^{D} is C₁₋₂ alkyl e.g. methyl. In a third embodiment, R^{D} is hydroxy. In a fourth embodiment, R^{D} is fluoro. In a fifth embodiment, R^{D} is C₁₋₂ alkoxy e.g. OMe.

In one embodiment, both R^{C} and R^{D} are H.

In another embodiment, R^{C} and R^{D} may join to form a C₃₋₅ cycloalkyl ring, such as a cyclopropyl ring.

In one embodiment, the compound of formula (I) is:

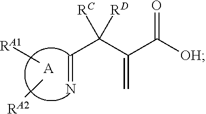

or a pharmaceutically acceptable salt and/or solvate thereof;
wherein A, R^{A1}, R^{A2}, R^{C} and R^{D} are as defined elsewhere herein. The carbon-carbon double bond in this structure is referred to as “exo”.

In another embodiment, the compound of formula (I) is:

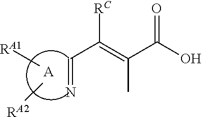

or a pharmaceutically acceptable salt and/or solvate thereof;
wherein A, R^{A1}, R^{A2} and R^{C} are as defined elsewhere herein. The carbon-carbon double bond in this structure is referred to as “endo”.

In the endo embodiment, the double bond may be cis or trans such that both of the following moieties are covered:

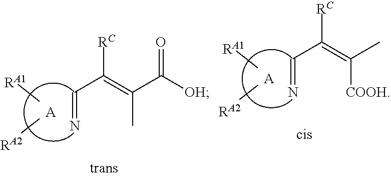

Similarly, as used herein, the following structure:

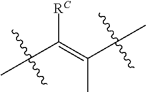

encompasses both cis and trans isomers:

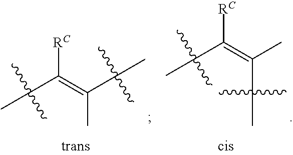

Suitably, the endo double bond in the compound of formula (I) is trans.

The total number of carbon atoms in groups R^{A1} and R^{A2} taken together including their optional substituents is 6-14 such as 6-12, suitably, 7-12 or 8-12 e.g. 6-10, 7-10 or 8-10.

In an embodiment, R^{A2} is absent and the total number of carbon atoms in group R^{A1} including any optional substituents is 7-12 or 8-12, or 6-10, 7-10 or 8-10.

In one embodiment, the compound of formula (I) is selected from the group consisting of:
2-((3-(6-butoxypyridin-3-yl)-1,2,4-oxadiazol-5-yl)methyl)acrylic acid;
2-((3-(5-butoxypyridin-2-yl)-1,2,4-oxadiazol-5-yl)methyl)acrylic acid;
2-((3-(3-2-((3-(5-butoxy-3-chloropyridin-2-yl)-1,2,4-oxadiazol-5-yl)methyl)acrylic acid; and
chloro-5-(4-fluorophenoxy)pyridin-2-yl)-1,2,4-oxadiazol-5-yl)methyl)acrylic acid;

or a pharmaceutically acceptable salt and/or solvate thereof.

The compounds of the invention may be prepared by the general methods described herein or as described in the Examples.

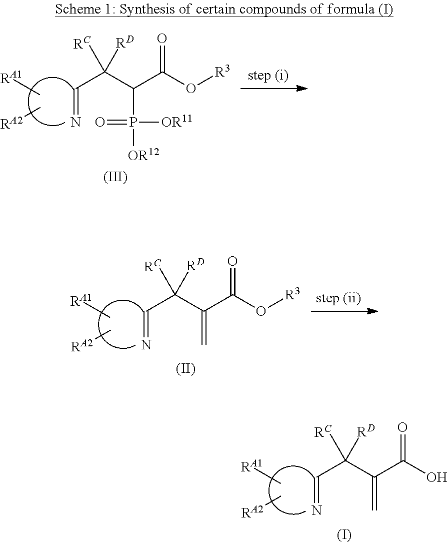

A, R^{A1}, R^{A2}, R^{C} and R^{D} are defined elsewhere herein. The synthesis of compounds of formula (III) is described elsewhere herein. R¹¹, R¹² and R³ independently represent C₁₋₄ alkyl optionally substituted with halo.

Step (i): compounds of formula (III) undergo a condensation reaction with formaldehyde or a formaldehyde equivalent thereof, e.g., paraformaldehyde, to give α,β-unsaturated esters of formula (II).

Step (ii): compounds of formula (II) are hydrolysed under standard acid or base hydrolysis conditions, e.g., TFA in DCM when R³ is tert-butyl, to give the compound of formula (I).

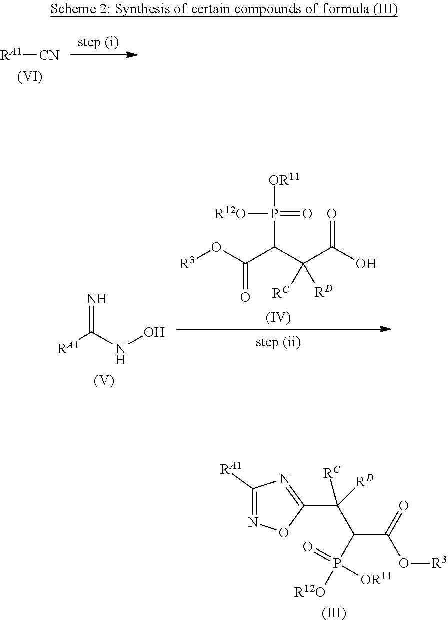

R¹¹, R¹² and R³ are defined in Scheme 1 above, R^{A1}, R^{C} and R^{D} are defined elsewhere herein, and R^{A2} is absent.

Step (i): Nitriles (VI) are converted to amidoximes (V) using hydroxylamine hydrochloride in the presence of sodium bicarbonate.

Step (ii): Certain compounds of formula (III) may be prepared by reacting amidoxime (V) with acid (IV), an example preparation for which is shown in the example section below, in the presence of a coupling agent such as T3P and a base such as NEt₃.

Compounds of formula (I) may be accessed from compounds of formula (III) as described in Scheme 1.

The skilled person will appreciate that protecting groups may be used throughout the synthetic scheme described above to give protected derivatives of any of the above compounds or generic formulae. Protective groups and the means for their removal are described in “Protective Groups in Organic Synthesis”, by Theodora W. Greene and Peter G. M. Wuts, published by John Wiley & Sons Inc; 4th Rev Ed., 2006, ISBN-10:0471697540. Examples of nitrogen protecting groups include tert-butyloxycarbonyl (BOC), 9-fluorenylmethyloxycarbonyl (Fmoc), acetyl (Ac), benzyl (Bn) and para-methoxy benzyl (PMB). Examples of oxygen protecting groups include acetyl (Ac), methoxymethyl (MOM), para-methoxybenzyl (PMB), benzyl, tert-butyl, methyl, ethyl, tetrahydropyranyl (THP), and silyl ethers and esters (such as trimethylsilyl (TMS), tert-butyldimethylsilyl (TBDMS), tri-iso-propylsilyloxymethyl (TOM), and triisopropylsilyl (TIPS) ethers and esters).

Thus, in one embodiment there is provided a process for preparing the compound of formula (I):

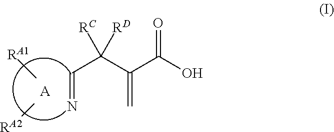

or a salt, such as a pharmaceutically acceptable salt thereof which comprises hydrolysing the ester moiety in a compound of formula (II):

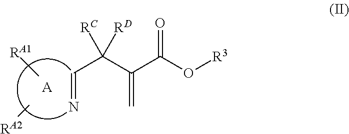

or a salt thereof,
wherein

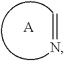

R^{A1}, R^{A2}, R^{C}, R^{D} and R³ are defined elsewhere herein.

In one embodiment there is provided a process for preparing a compound of formula (II):

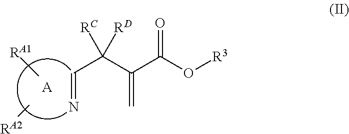

or a salt thereof which comprises reacting a compound of formula (III):

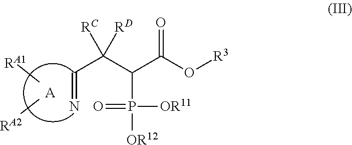

or a salt thereof;
with formaldehyde or an equivalent thereof,
wherein

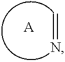

R^{A1}, R^{A2}, R^{C}, RD, R³, R¹¹ and R¹² are defined elsewhere herein.

In one embodiment there is provided a compound of formula (II):

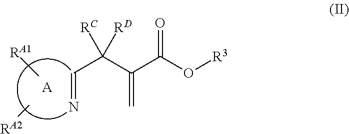

or salt thereof, wherein

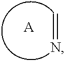

R^{A1}, R^{A2}, R^{C}, R^{D} and R³ are defined elsewhere herein.

In one embodiment there is provided a compound of formula (III):

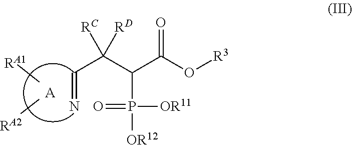

or salt thereof, wherein

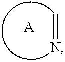

R^{A1}, R^{A2}, R^{C}, RD, R¹¹, R¹² and R³ are defined elsewhere herein.

In one embodiment, the molecular weight of the compound of formula (I) is 150 Da-500 Da, especially 200 Da-350 Da.

It will be appreciated that for use in therapy the salts of the compounds of formula (I) should be pharmaceutically acceptable. Suitable pharmaceutically acceptable salts will be apparent to those skilled in the art. Pharmaceutically acceptable salts include basic addition salts such as sodium, potassium, calcium, aluminium, zinc, magnesium and other metal salts. Pharmaceutically acceptable salts may also be formed with organic bases e.g. with ammonia, meglumine, tromethamine, piperazine, arginine, choline, diethylamine, benzathine or lysine. Other pharmaceutically acceptable salts include a trifluoroacetic acid salt. Suitably, the pharmaceutically acceptable salt is a tromethamine salt. Thus, in one embodiment there is provided a compound of formula (I) in the form of a pharmaceutically acceptable salt. Alternatively, there is provided a compound of formula (I) in the form of a free acid. When the compound contains a basic group as well as the free acid it may be Zwitterionic.

In one embodiment, the compound of formula (I) or a pharmaceutically acceptable salt and/or solvate thereof is a compound of formula (I).

In one embodiment, the compound of formula (I) or a pharmaceutically acceptable salt and/or solvate thereof is a pharmaceutically acceptable salt of a compound of formula (I).

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water). Suitably, the compound of formula (I) is not a solvate.

It is to be understood that the present invention encompasses all isomers of compounds of formula (I) including all geometric, tautomeric and optical forms, and mixtures thereof (e.g. racemic mixtures). Insofar as described herein, e.g., in claim 1, certain specific structural isomers are provided as part of the invention. In particular, the invention extends to all tautomeric forms of the compounds of formula (I). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in positron emission topography (PET) studies for examining substrate receptor occupancy.

In one embodiment, the compounds of formula (I) are provided in a natural isotopic form. In one embodiment, the compounds of formula (I) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (I). In one embodiment, the atoms of the compounds of formula (I) are in an isotopic form which is not radioactive. In one embodiment, one or more atoms of the compounds of formula (I) are in an isotopic form which is radioactive. Suitably radioactive isotopes are stable isotopes. Suitably the unnatural variant isotopic form is a pharmaceutically acceptable form.

In one embodiment, a compound of formula (I) is provided whereby a single atom of the compound exists in an unnatural variant isotopic form. In another embodiment, a compound of formula (I) is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### Therapeutic Indications

Compounds of formula (I) are of use in therapy, particularly for treating or preventing an inflammatory disease or a disease associated with an undesirable immune response. As shown in Biological Example 1 below, example compounds of formula (I) reduced cytokine release more effectively than 4-octyl itaconate, 2-(2-chlorobenzyl)acrylic acid and/or monomethyl fumarate in IL-1β and/or IL-6, as demonstrated by lower IC₅₀ values. As shown in Biological Example 2 below, certain example compounds of formula (I) showed improved activity compared to 2-(2-chlorobenzyl)acrylic acid and 4-octyl itaconate, as demonstrated by their lower EC₅₀ and/or higher Eₘₐₓ values for NRF2 activation. Thus, the compounds may be expected to have utility in the treatment of diseases wherein such activity may be beneficial (such as multiple sclerosis, psoriasis and chronic obstructive pulmonary disease: Cuadrado et al., Nat. Rev. Drug Discov. 2019, 18, 295-317). As shown in Biological Example 3 below, certain example compounds of formula (I) showed improved metabolic stabilities compared to 4-octyl itaconate in both human and mouse species. Certain compounds showed improved metabolic stabilities compared to 2-(2-chlorobenzyl)acrylic acid in human hepatocytes.

Thus, in a further aspect, the present invention provides a compound of formula (I) or a pharmaceutically acceptable salt and/or solvate thereof as defined herein, for use as a medicament.

Also provided is a pharmaceutical composition comprising a compound of formula (I) or a pharmaceutically acceptable salt and/or solvate thereof as defined herein. Thus, in a further aspect, the present invention provides a pharmaceutical composition comprising a compound of formula (I) or a pharmaceutically acceptable salt and/or solvate thereof as defined herein, for use as a medicament.

In a further aspect, the present invention provides a compound of formula (I) or a pharmaceutically acceptable salt and/or solvate thereof as defined herein, for use in treating or preventing an inflammatory disease or a disease associated with an undesirable immune response.

In a further aspect, the present invention provides a pharmaceutical composition as defined herein, for use in treating or preventing an inflammatory disease or a disease associated with an undesirable immune response.

In a further aspect, the present invention provides the use of a compound of formula (I) or a pharmaceutically acceptable salt and/or solvate thereof as defined herein, in the manufacture of a medicament for treating or preventing an inflammatory disease or a disease associated with an undesirable immune response.

In a further aspect, the present invention provides the use of a pharmaceutical composition as defined herein, in the manufacture of a medicament for treating or preventing an inflammatory disease or a disease associated with an undesirable immune response.

In a further aspect, the present invention provides a method of treating or preventing an inflammatory disease or a disease associated with an undesirable immune response, which comprises administering a compound of formula (I) or a pharmaceutically acceptable salt and/or solvate thereof as defined herein.

In a further aspect, the present invention provides a method of treating or preventing an inflammatory disease or a disease associated with an undesirable immune response, which comprises administering a pharmaceutical composition as defined herein.

For all aspects of the invention, suitably the compound is administered to a subject in need thereof, wherein the subject is suitably a human subject.

In one embodiment is provided a compound of formula (I) or a pharmaceutically acceptable salt and/or solvate thereof as defined herein, for use in treating an inflammatory disease or disease associated with an undesirable immune response. In one embodiment of the invention is provided the use of a compound of formula (I) or a pharmaceutically acceptable salt and/or solvate thereof as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (I) or a pharmaceutically acceptable salt and/or solvate thereof as defined herein.

In one embodiment is provided a pharmaceutical composition as defined herein, for use in treating an inflammatory disease or disease associated with an undesirable immune response. In one embodiment of the invention is provided the use of a pharmaceutical composition as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a pharmaceutical composition as defined herein.

In one embodiment is provided a compound of formula (I) or a pharmaceutically acceptable salt and/or solvate thereof as defined herein, for use in preventing an inflammatory disease or a disease associated with an undesirable immune response. In one embodiment of the invention is provided the use of a compound of formula (I) or a pharmaceutically acceptable salt and/or solvate thereof as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (I) or a pharmaceutically acceptable salt and/or solvate thereof as defined herein.

In one embodiment is provided a pharmaceutical composition as defined herein, for use in preventing an inflammatory disease or a disease associated with an undesirable immune response. In one embodiment of the invention is provided the use of a pharmaceutical composition as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a pharmaceutical composition as defined herein.

In one embodiment is provided a compound of formula (I) or a pharmaceutically acceptable salt and/or solvate thereof as defined herein, or a pharmaceutical composition as defined herein, for use in treating or preventing an inflammatory disease. In one embodiment of the invention is provided the use of a compound of formula (I) or a pharmaceutically acceptable salt and/or solvate thereof as defined herein, or a pharmaceutical composition as defined herein, in the manufacture of a medicament for treating or preventing an inflammatory disease. In one embodiment of the invention is provided a method of treating or preventing an inflammatory disease, which comprises administering a compound of formula (I) or a pharmaceutically acceptable salt and/or solvate thereof as defined herein, or a pharmaceutical composition as defined herein.

In one embodiment is provided a compound of formula (I) or a pharmaceutically acceptable salt and/or solvate thereof as defined herein, or a pharmaceutical composition as defined herein, for use in treating or preventing a disease associated with an undesirable immune response. In one embodiment of the invention is provided the use of a compound of formula (I) or a pharmaceutically acceptable salt and/or solvate thereof as defined herein, or a pharmaceutical composition as defined herein, in the manufacture of a medicament for treating or preventing a disease associated with an undesirable immune response. In one embodiment of the invention is provided a method of treating or preventing a disease associated with an undesirable immune response, which comprises administering a compound of formula (I) or a pharmaceutically acceptable salt and/or solvate thereof as defined herein, or a pharmaceutical composition as defined herein.

An undesirable immune response will typically be an immune response which gives rise to a pathology i.e. is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenia purpura, thrombotic thrombocytopeni purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-) encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C(HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In another embodiment, the disease is selected from the group consisting of spondyloarthrapathies, polymyalgia rheumatica and erosive osteoarthritis of the hands.

In one embodiment, the disease is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

The link between certain diseases listed herein and targeting IL-1beta, IL-6 or NRF2 is known from the literature as described below inter alia. Thus, compounds of formula (I) (which compounds target IL-1beta, IL-6 and/or NRF2 as shown in the Biological Example section) are expected to have utility in the treatment of such diseases.

In particular, the literature provides support for targeting IL-1beta, IL-6 and/or NRF2 and treating at least rheumatoid arthritis (Giacomelli et al. 2016); psoriatic arthritis (Al-Hwas et al., 2022); systemic lupus erythematosus (Sung et al. 2020); multiple sclerosis (Mendiola et al. 2018); psoriasis (Tsuji et al. 2020); Crohn's disease (Piotrowska et al. 2021); ulcerative colitis (Liso et al. 2022); juvenile idiopathic arthritis (Toplak et al. 2018); uveitis (Fabiani et al. 2017); spondyloarthrapathies (Keller et al. 2003); ankylosing spondylitis (Ferrándiz et al. 2018); polymyalgia rheumatica (Weyand et al. 1994); erosive osteoarthritis of the hands (Fioravanti et al. 2019); Lupus nephritis (Italiani et al. 2018); Parkinson's disease (Karpenko et al. 2018); inflammatory bowel disease (Friedrich et al. 2021); celiac disease (Nasserinejad et al., 2019); dermatomyositis (Authier et al. 1997); hidradenitis suppurativa (Witte-Händel et al. 2019); Sjogren's syndrome (Bårdsen et al. 2019); giant cell arteritis (temporal arteritis) (Ly et al. 2014); systemic juvenile idiopathic arthritis (Still's disease) (Toplak et al. 2018); familial Mediterranean fever (FMF) (Migita et al. 2015); tumour necrosis factor (TNF) receptor-associated periodic fever syndrome (TRAPS) (Dandekar et al. 2015); hyperimmunoglobulinaemia D with periodic fever syndrome (HIDS) (Kaneko et al. 2019); cryopyrin-associated periodic syndromes (CAPS) (Dhimolea 2011); Aicardi-Goutières syndrome (Takanohashi et al. 2013); and spondyloenchondrodysplasia (Lindahl et al. 2022).

Thus in one embodiment, the disease is selected from the group consisting of rheumatoid arthritis; psoriatic arthritis; systemic lupus erythematosus; multiple sclerosis; psoriasis; Crohn's disease; ulcerative colitis; juvenile idiopathic arthritis; uveitis; spondyloarthrapathies; ankylosing spondylitis; temporal arteritis; polymyalgia rheumatica; erosive osteoarthritis of the hands; Lupus nephritis; Parkinson's disease; inflammatory bowel disease; celiac disease; dermatomyositis; hidradenitis suppurativa; Sjogren's syndrome; giant cell arteritis (temporal arteritis); systemic juvenile idiopathic arthritis (Still's disease); familial Mediterranean fever (FMF); tumour necrosis factor (TNF) receptor-associated periodic fever syndrome (TRAPS); hyperimmunoglobulinaemia D with periodic fever syndrome (HIDS); cryopyrin-associated periodic syndromes (CAPS); Aicardi-Goutières syndrome; and spondyloenchondrodysplasia.

In one embodiment, the disease is multiple sclerosis. In one embodiment, the disease is psoriasis. In one embodiment, the disease is asthma. In one embodiment, the disease is chronic obstructive pulmonary disease. In one embodiment, the disease is systemic lupus erythematosus. In one embodiment, the disease is rheumatoid arthritis. In one embodiment, the disease is psoriatic arthritis. In one embodiment, the disease is Parkinson's disease. In one embodiment, the disease is Crohn's disease. In one embodiment, the disease is ulcerative colitis. In one embodiment, the disease is juvenile idiopathic arthritis. In one embodiment, the disease is uveitis. In one embodiment, the disease is spondyloarthrapathies. In one embodiment, the disease is ankylosing spondylitis. In one embodiment, the disease is temporal arteritis. In one embodiment, the disease is polymyalgia rheumatica. In one embodiment, the disease is erosive osteoarthritis of the hands.

In one embodiment, the disease is Lupus nephritis. In one embodiment, the disease is inflammatory bowel disease. In one embodiment, the disease is celiac disease. In one embodiment, the disease is dermatomyositis. In one embodiment, the disease is hidradenitis suppurativa.

### Administration

The compound of formula (I) is usually administered as a pharmaceutical composition. Thus, in one embodiment, is provided a pharmaceutical composition comprising a compound of formula (I) and one or more pharmaceutically acceptable diluents or carriers. Also provided is a pharmaceutical composition comprising a compound of formula (I) or a pharmaceutically acceptable salt and/or solvate thereof as defined herein. Such a pharmaceutical composition contains the compound of formula (I) and a pharmaceutically acceptable carrier or excipient.

The compound of formula (I) may be administered by any convenient method, e.g. by oral, parenteral, buccal, sublingual, nasal, rectal, intrathecal or transdermal administration, and the pharmaceutical compositions adapted accordingly.

The compound of formula (I) may be administered topically to the target organ e.g. topically to the eye, lung, nose or skin. Hence the invention provides a pharmaceutical composition comprising a compound of formula (I) optionally in combination with one or more topically acceptable diluents or carriers.

A compound of formula (I) which is active when given orally can be formulated as a liquid or solid, e.g. as a syrup, suspension, emulsion, tablet, capsule or lozenge.

A liquid formulation will generally consist of a suspension or solution of the compound of formula (I) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (I) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (I) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (−) 10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen (R), specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (I) is formulated with a carrier such as sugar and acacia, tragacanth, or gelatine and glycerine.

Compositions suitable for transdermal administration include ointments, gels and patches.

The composition may contain from 0.1% to 100% by weight, for example from 10 to 60% by weight, of the compound of formula (I), depending on the method of administration. The composition may contain from 0% to 99% by weight, for example, 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (I), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (I) is used in combination with a further therapeutic agent or agents. When the compound of formula (I) is used in combination with other therapeutic agents, the compounds may be administered either sequentially or simultaneously by any convenient route. Alternatively, the compounds may be administered separately.

Therapeutic agents which may be used in combination with the present invention include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibition (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), anti-thymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BLyS, B lymphocyte stimulator) inhibitors (e.g. belimumab, blisibimod), other B cell targeted therapy including fusion proteins targeting both APRIL (A PRoliferation-Inducing Ligand) and BLyS (e.g. atacicept), PI3K inhibitors including pan-inhibitors or those targeting the p110δ and/or p110γ containing isoforms (e.g. idelalisib, copanlisib, duvelisib), interferon α receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4β1 and/or α4β7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

Compounds of formula (I) may display one or more of the following desirable properties:
low IC₅₀ values for inhibiting release of cytokines e.g. IL-1β and/or IL-6, from cells;
low EC₅₀ and/or high Eₘₐₓ values for activating the enzyme NQO1 or the NRF2 pathway;
enhanced efficacy through improved metabolic stability and/or augmented maximum response;
reduced dose and dosing frequency through improved pharmacokinetics, especially as a result of enhanced stability in hepatocytes;
improved oral systemic bioavailability;
reduced plasma clearance following intravenous dosing;
improved metabolic stability e.g. as demonstrated by improved stability in plasma and/or hepatocytes;
augmented cell permeability;
enhanced aqueous solubility;
good tolerability, for example, by limiting the flushing and/or gastrointestinal side effects provoked by oral DMF (Hunt T. et al., 2015; WO2014/152494A1, incorporated herein by reference), possibly by reducing or eliminating HCA2 activity;
low toxicity at the relevant therapeutic dose;
distinct anti-inflammatory profiles resulting from varied electrophilicities, leading to differential targeting of the cysteine proteome (van der Reest J. et al., 2018) and, therefore, modified effects on gene activation;
glutathione-sparing actions;
avoiding the oncometabolite fumaric acid (Kulkarni R. A. et al., 2019);
improved physical form (solid) or higher melting point.

### Abbreviations

Ac acetyl
aq. aqueous
ATG anti-thymocyte
β beta
BBFO broadband fluorine observe
BEH ethylene bridged hybrid
Bn benzyl
BOC tert-butyloxycarbonyl
CSH charged surface hybrid
d doublet
DAD diode array detector
DCM dichloromethane
DMF dimethyl fumarate
DMI dimethyl itaconate
DMSO dimethyl sulfoxide
Et ethyl
ES+ electrospray
FBS fetal bovine serum
g gram(s)
GSH glutathione
h hour(s)
HATU 1-[bis(dimethylamino)methylene]-1H-1,2,3-triazolo[4,5-b]pyridinium 3-oxid hexafluorophosphate
HFC hydrofluorocarbon
HPLC high-performance liquid chromatography
IL interleukin
IPA isopropyl alcohol
K kelvin
LCMS liquid chromatography-mass spectrometry
LPS lipopolysaccharide
m multiplet
M molar concentration/molar mass
m/z mass to charge ratio
Me methyl
(M)Hz (mega)hertz
mg milligram
min(s) minute(s)
mL millilitre
mm millimetre
MMF monomethyl fumarate
mmol millimole
MOM methoxymethyl
MS mass spectrometry
MSD mass selective detector
MTBE methyl tert-butyl ether
nm nanometre
NMR nuclear magnetic resonance
NQO1 NAD(P)H dehydrogenase [quinone] 1
NRF2 nuclear factor (erythroid-derived 2)-like 2
NSAIDS non-steroidal anti-inflammatory drugs
PAH pulmonary arterial hypertension
PBS phosphate buffered saline
PDA photodiode array
PDE4 phosphodiesterase-4
PET positron emission topography
PMB para-methoxybenzyl
PUVA psoralen ultraviolet irradiation
4OI 4-octyl itaconic acid
rpm revolutions per minute
RT room temperature
S singlet
sat. saturated
t triplet
T3P propylphosphonic anhydride
TBDMS tert-butyldimethylsilyl
tBu tert-butyl
Tf triflyl
TFA trifluoroacetic acid
THF tetrahydrofuran
TIPS triisopropylsilyl
TLR Toll-like receptor
TMS trimethylsilyl
TNF tumour necrosis factor
Ts tosyl
μL microlitre
μM micromolar
μmol micromole
UPLC ultra performance liquid chromatography
UV ultra violet
VEGF vascular endothelial growth factor
VWD variable wavelength detector
wt. weight
XRPD X-Ray Powder Diffraction
° C. degrees centigrade

### Examples

Analytical Equipment Thin layer chromatography (TLC) was performed on silica gel plates (GF254, glass, silica gel size: 400˜600 mesh). Spots were visualized by UV light (214 and 254 nm) or color reagents (iodine, KMnO4 aq.).

Bruker 400 MHz Avance III spectrometer fitted with a BBFO 5 mm probe, or a Bruker 500 MHz Avance III HD spectrometer equipped with a Bruker 5 mm SmartProbe™. 1H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration.

LCMS/UPLC Instrument Details

| System | Instrument Name | LC Detector | ELS detector | Mass detector |
|---|---|---|---|---|
| 1 (basic) | Agilent LCMS 1200 | G1315D DAD | 380 ELSD | Agilent G6120B |
| 2 (acidic) | Agilent LCMS 1200 | G1315C DAD | 380 ELSD | Agilent G6110A |
| 3 (UPLC) | Waters H-Class | Acquity PDA | n/a | Acquity QDa |
| 4 (LCMS) | Agilent LCMS 1260 | G7117C DAD | n/a | Agilent G6125B |

LCMS/HPLC/UPLC Method Details

| | | | | | | Column | Flow |
|---|---|---|---|---|---|---|---|
| Method | | | | | Mass | Temp. | Rate |
| Name | Solvent System | Column | Gradient | UV range | Range | ° C. | ml/min |
| A (basic | A) water + 10 mM | Waters X-Bridge | From 95:5 to 0:100 in 1.6 min, | 190-400 | 100-1800 | 40 | 2.0 |
| LCMS) | NH₄HCO₃ | C18 (50 mm × 4.6 | 0:100 for 1.4 min, from 0:100 to | nm | amu | | |
| | B) acetonitrile | mm × 3.5 μm) | 95:5 in 0.1 min, 95:5 for 0.7 min. | | | | |
| B (acidic | A) water + 0.05% | Waters X-Bridge | From 95:5 to 0:100 in 1.6 min, | 190-400 | 100-1100 | 40 | 2.0 |
| LCMS) | TFA | C18 (50 mm × 4.6 | 0:100 for 1.4 min, from 0:100 to | nm | amu | | |
| | B) acetonitrile + | mm × 3.5 μm) | 95:5 in 0.05 min, 95:5 for 0.7 min | | | | |
| | 0.05% TFA | | | | | | |
| C (acidic | A) water + | Halo C18 (30 mm × | From 95:5 to 0:100 in 0.8 min, | 190-400 | 100-1100 | 40 | 3.0 |
| LCMS) | 0.05% TFA | 4.6 mm × 2.7 μm) | 0:100 for 0.4 min, from 0:100 to | nm | amu | | |
| | B) acetonitrile + | | 95:5 in 0.01 min, 95:5 for 0.2 min | | | | |
| | 0.05% TFA | | | | | | |
| D (acidic | A) water + 0.1% | Waters ACQUITY | From 98:2 to 0:100 in 2.5 min, | 210-400 | 100-1100 | 40 | 0.77 |
| UPLC) | formic acid | UPLC ® CSH C18, | 0:100 for 0.5 min | nm | amu | | |
| | B) acetonitrile | 1.7 μm, 2.1 × 30 | | | | | |
| | | mm | | | | | |
| E (basic | A) water + 0.2% | Waters ACQUITY | From 98:2 to 0:100 in 2.5 min, | 210-400 | 100-1100 | 40 | 0.77 |
| UPLC) | ammonia | UPLC ® BEH C18, | 0:100 for 0.5 min | nm | amu | | |
| | B) acetonitrile | 1.7 μm, 2.1 × 30 | | | | | |
| | | mm | | | | | |
| F (acidic | A) water + 0.1% | Waters Cortecs | From 98:2 to 0:100 in 2.5 min, | 260 nm +/− | 100-1100 | 40 | 1.35 |
| LCMS) | formic acid | C18, 30 × 2.1 mm, | 0:100 for 0.5 min | 90 nm | amu | | |
| | B) acetonitrile | 2.7 μm | | | | | |
| G (basic | A) water + 0.2% | Phenomenex Evo | From 98:2 to 0:100 in 2.5 min, | 260 nm +/− | 100-1100 | 40 | 1.35 |
| LCMS) | ammonia | C18, 30 × 2.1 mm, | 0:100 for 0.5 min | 90 nm | amu | | |
| | B) acetonitrile | 2.6 μm | | | | | |

### General Methods

All starting materials are commercially available unless otherwise stated. Unless otherwise stated all reactions were stirred.

### Synthesis of Intermediates

### Intermediate 1:4-(tert-butoxy)-3-(diethoxyphosphoryl)-4-oxobutanoic acid

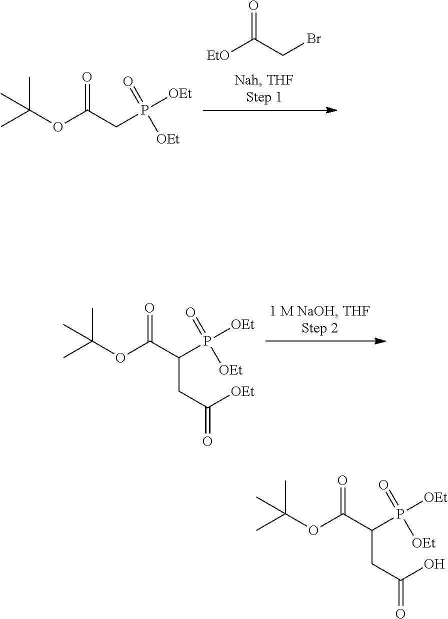 [SMILES: CCOC(=O)CBr ; CCOP(=O)(CC(=O)OC(C)(C)C)OCC ; CCOC(=O)CC(C(=O)OC(C)(C)C)P(=O)(OCC)OCC ; CCOP(=O)(OCC)C(CC(=O)O)C(=O)OC(C)(C)C]

### Step 1

Sodium hydride (60 wt % dispersion in mineral oil, 9.0 g, 225 mmol) was added portionwise to a solution of tert-butyl 2-(diethoxyphosphoryl)acetate (50 mL, 213 mmol) in THF (500 mL) at 0° C. The mixture was stirred for 15 min before ethyl bromoacetate (23 mL, 210 mmol) was added dropwise. The mixture was stirred for 1 h then quenched with sat. aq. NH₄Cl (100 mL) and extracted with EtOAc (3×100 mL). The combined organic phases were washed with brine (300 mL), dried (MgSO₄) and concentrated to afford 1-(tert-butyl) 4-ethyl 2-(diethoxyphosphoryl) succinate (77.1 g, 182 mmol, 80% purity) as a colourless oil. ¹H NMR (400 MHz, DMSO-d6) δ 4.13-4.01 (m, 6H), 3.28 (ddd, J=23.8, 11.3, 3.9 Hz, 1H), 2.78 (ddd, J=17.2, 11.3, 8.2 Hz, 1H), 2.64 (ddd, J=17.1, 8.5, 4.0 Hz, 1H), 1.40 (s, 9H), 1.28-1.21 (m, 6H), 1.18 (t, J=7.1 Hz, 3H). LCMS (System 3, Method D) m/z 361.2 (M+Na)⁺ (ES⁺).

### Step 2

An aqueous solution of sodium hydroxide (1 M, 250 mL, 250 mmol) was added to a solution of 1-(tert-butyl) 4-ethyl 2-(diethoxyphosphoryl) succinate (77.1 g, 182 mmol, 80% purity) in THF (250 mL). The mixture was stirred at RT for 16 h. The mixture was partially concentrated to ca. 250 mL, then extracted with EtOAc (3×100 mL). The aqueous phase was acidified to pH 1 with conc. HCl and extracted with EtOAc (3×100 mL). The combined organic phases were washed with brine (250 mL), dried (MgSO₄) and concentrated. The residue was triturated with hexane (300 mL) and the resulting solid collected by filtration to afford 4-(tert-butoxy)-3-(diethoxyphosphoryl)-4-oxobutanoic acid (53.0 g, 0.15 mol, 90% purity) as a white solid. ¹H NMR (400 MHz, DMSO-d6) δ 12.44 (s, 1H), 4.11-3.99 (m, 4H), 3.22 (ddd, J=23.7, 11.5, 3.7 Hz, 1H), 2.73 (ddd, J=17.3, 11.5, 7.6 Hz, 1H), 2.56 (ddd, J=17.3, 8.6, 3.7 Hz, 1H), 1.40 (s, 9H), 1.25 (dt, J=8.3, 7.0 Hz, 6H). ³¹P NMR (162 MHz, DMSO-d6) δ 21.88. LCMS (System 3, Method D) m/z 333.2 (M+Na)⁺ (ES⁺).

### Synthesis of Examples

### Example 1:2-((3-(6-butoxypyridin-3-yl)-1,2,4-oxadiazol-5-yl)methyl)acrylic acid

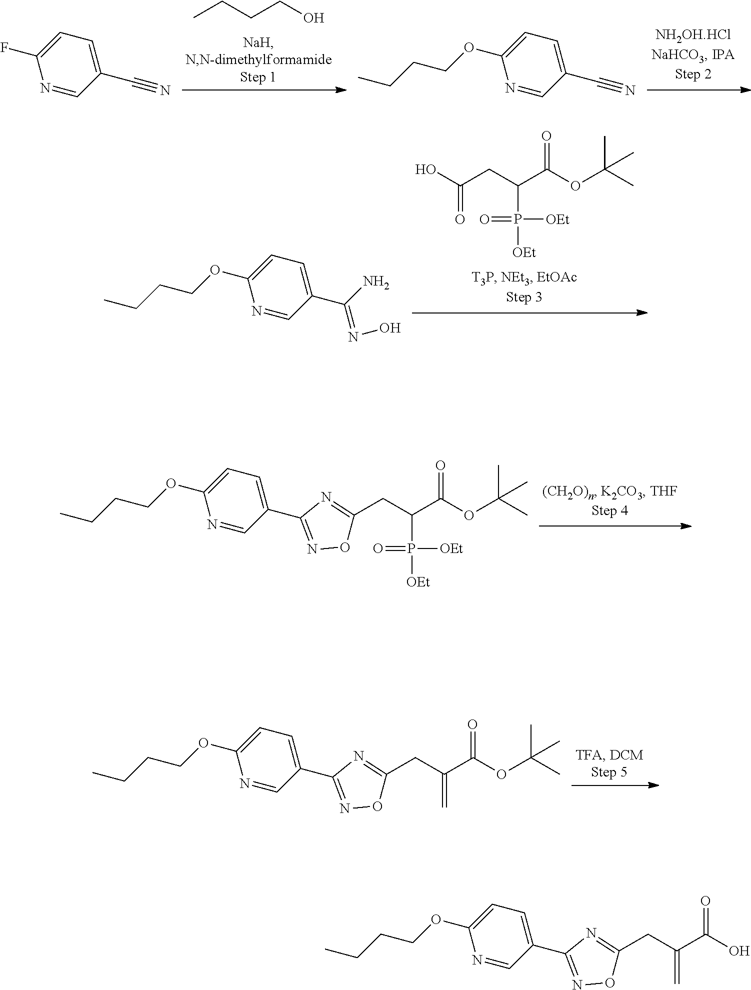

### Step 1

To a suspension of sodium hydride (60 wt % dispersion in mineral oil, 164 mg, 4.1 mmol) in N,N-dimethylformamide (6.74 mL) was added butan-1-ol (304 mg, 0.37 mL, 4.10 mmol) followed by 6-fluoronicotinonitrile (0.5 g, 4.10 mmol) all at once at RT. The reaction was left to stir at RT overnight for 16 h. The reaction was quenched with water (25 mL) and the mixture was extracted with EtOAc (3×30 mL), the combined organic layers were washed with brine (2×30 mL), dried over Na₂SO₄, filtered and concentrated. The crude product was purified by chromatography on silica gel (0-50% MTBE/iso-hexane) to afford 6-butoxynicotinonitrile (0.54 g, 3.1 mmol, 99% purity) as a light yellow oil. ¹H NMR (400 MHz, DMSO-d6) δ 8.68 (d, J=2.5 Hz, 1H), 8.20-8.09 (m, 1H), 6.99 (d, J=8.7 Hz, 1H), 4.33 (t, J=6.6 Hz, 2H), 1.76-1.66 (m, 2H), 1.48-1.35 (m, 2H), 0.92 (t, J=7.4 Hz, 3H). LCMS (System 3, Method D) m/z 177.1 (M+H)⁺ (ES⁺).

### Step 2

A suspension of hydroxylamine hydrochloride (321 mg, 4.62 mmol) and sodium bicarbonate (647 mg, 7.7 mmol) in IPA (6.7 mL) was stirred at RT for 15 mins then 6-butoxynicotinonitrile (0.54 g, 3.1 mmol) was added dropwise as a solution in IPA (2 mL) over 5 mins. The reaction was heated to 85° C. for 72 h. The reaction was cooled to RT and the insoluble inorganics were filtered off. The filtrate was concentrated and excess IPA was co-evaporated with toluene (2×10 mL) to give a white solid which was triturated with iso-hexane (15 mL) and re-filtered to give (Z)-6-butoxy-N′-hydroxynicotinimidamide (0.65 g, 2.2 mmol, 72% purity) as a white solid. ¹H NMR (400 MHz, DMSO-d6) 0 9.61 (s, 1H), 8.41 (d, J=2.5 Hz, 1H), 7.92 (dd, J=8.6, 2.5 Hz, 1H), 6.78 (d, J=8.7 Hz, 1H), 5.85 (s, 2H), 4.26 (t, J=6.6 Hz, 2H), 1.75-1.62 (m, 2H), 1.48-1.35 (m, 2H), 0.92 (t, J=7.4 Hz, 3H). LCMS (System 3, Method D) m/z 210.6 (M+H)⁺ (ES⁺).

### Step 3

To a solution of (Z)-6-butoxy-N′-hydroxynicotinimidamide (0.65 g, 2.2 mmol, 72% purity) and 4-(tert-butoxy)-3-(diethoxyphosphoryl)-4-oxobutanoic acid (Intermediate 1, 881 mg, 2.84 mmol) in EtOAc (1.42 mL) was added triethylamine (862 mg, 1.19 mL, 8.52 mmol) at RT. To the reaction was added 2,4,6-tripropyl-1,3,5,2,4,6-trioxatriphosphinane 2,4,6-trioxide (50 wt % in EtOAc, 4.52 g, 4.23 mL, 7.10 mmol) dropwise over 10 mins at RT then the reaction was heated to 80° C. overnight for 16 h. The reaction was cooled to RT and poured into ice water (30 mL). The mixture was extracted with EtOAc (3×20 mL). The combined organic layers were washed with aq. sat. NaHCO₃ (2×25 mL), brine (30 mL), dried over Na₂SO₄, filtered and then concentrated. The crude product was purified by chromatography on silica gel (0-100% MTBE/hexane) to afford tert-butyl 3-(3-(6-butoxypyridin-3-yl)-1,2,4-oxadiazol-5-yl)-2-(diethoxyphosphoryl) propanoate (0.94 g, 1.9 mmol, 98% purity) as a light yellow oil. ¹H NMR (400 MHz, DMSODMSO-d6) δ 8.73 (dd, J=2.4, 0.7 Hz, 1H), 8.19 (dd, J=8.7, 2.4 Hz, 1H), 6.99 (dd, J=8.7, 0.8 Hz, 1H), 4.33 (t, J=6.6 Hz, 2H), 4.16-4.06 (m, 4H), 3.67 (ddd, J=23.4, 10.7, 4.6 Hz, 1H), 3.46 (ddd, J=16.8, 10.7, 9.7 Hz, 1H), 3.39-3.32 (m, 1H), 1.76-1.68 (m, 2H), 1.48-1.35 (m, 11H), 1.29-1.22 (m, 6H), 0.93 (t, J=7.4 Hz, 3H). LCMS (System 3, Method D) m/z 506.5 (M+Na)⁺ (ES⁺).

### Step 4

To a suspension of tert-butyl 3-(3-(6-butoxypyridin-3-yl)-1,2,4-oxadiazol-5-yl)-2-(diethoxyphosphoryl) propanoate (0.94 g, 1.9 mmol) and potassium carbonate (317 mg, 2.29 mmol) in THF (9.55 mL) was added paraformaldehyde (90.5 mg, 2.86 mmol) all at once at RT. The reaction was heated to 50° C. for 3 h and then cooled to RT and poured into water (25 mL). The mixture was extracted with EtOAc (3×15 mL) and the combined organic layers were washed with brine (30 mL), dried over Na₂SO₄, filtered and then concentrated. The crude product was purified by chromatography on silica gel (0-50% MTBE/hexane) to afford tert-butyl 2-((3-(6-butoxypyridin-3-yl)-1,2,4-oxadiazol-5-yl)methyl) acrylate (0.59 g, 1.6 mmol, 99% purity) as a clear colourless oil. ¹H NMR (400 MHz, DMSODMSO-d6) δ 8.73 (dd, J=2.5, 0.8 Hz, 1H), 8.20 (dd, J=8.7, 2.4 Hz, 1H), 6.97 (dd, J=8.7, 0.8 Hz, 1H), 6.28 (d, J=1.2 Hz, 1H), 6.01-5.94 (m, 1H), 4.33 (t, J=6.6 Hz, 2H), 4.04 (s, 2H), 1.77-1.67 (m, 2H), 1.50-1.37 (m, 2H), 1.34 (s, 9H), 0.93 (t, J=7.4 Hz, 3H). LCMS (System 3, Method D) m/z 360.4 (M+H)⁺ (ES⁺).

### Step 5

To a stirred solution of tert-butyl 2-((3-(6-butoxypyridin-3-yl)-1,2,4-oxadiazol-5-yl)methyl) acrylate (0.59 g, 1.6 mmol) in DCM (6.42 mL) was added TFA (3.76 g, 2.54 mL, 32.9 mmol) all at once at RT. The reaction was stirred at RT for 18 h at which point the solvent was removed under reduced pressure. The crude product was purified by chromatography on silica gel (0-50% MTBE/isohexane) to afford 2-((3-(6-butoxypyridin-3-yl)-1,2,4-oxadiazol-5-yl)methyl)acrylic acid (380 mg, 1.2 mmol, 99% purity) as a white solid. ¹H NMR (400 MHz, DMSO-d6) δ 12.88 (s, 1H), 8.73 (dd, J=2.5, 0.8 Hz, 1H), 8.20 (dd, J=8.7, 2.4 Hz, 1H), 6.97 (dd, J=8.6, 0.8 Hz, 1H), 4.33 (t, J=6.6 Hz, 2H), 4.03 (s, 2H), 1.78-1.65 (m, 2H), 1.49-1.35 (m, 2H), 0.93 (t, J=7.4 Hz, 3H). LCMS (System 3, Method D) m/z 304 (M+H)⁺ (ES⁺).

### Example 2:2-((3-(5-butoxypyridin-2-yl)-1,2,4-oxadiazol-5-yl)methyl)acrylic acid

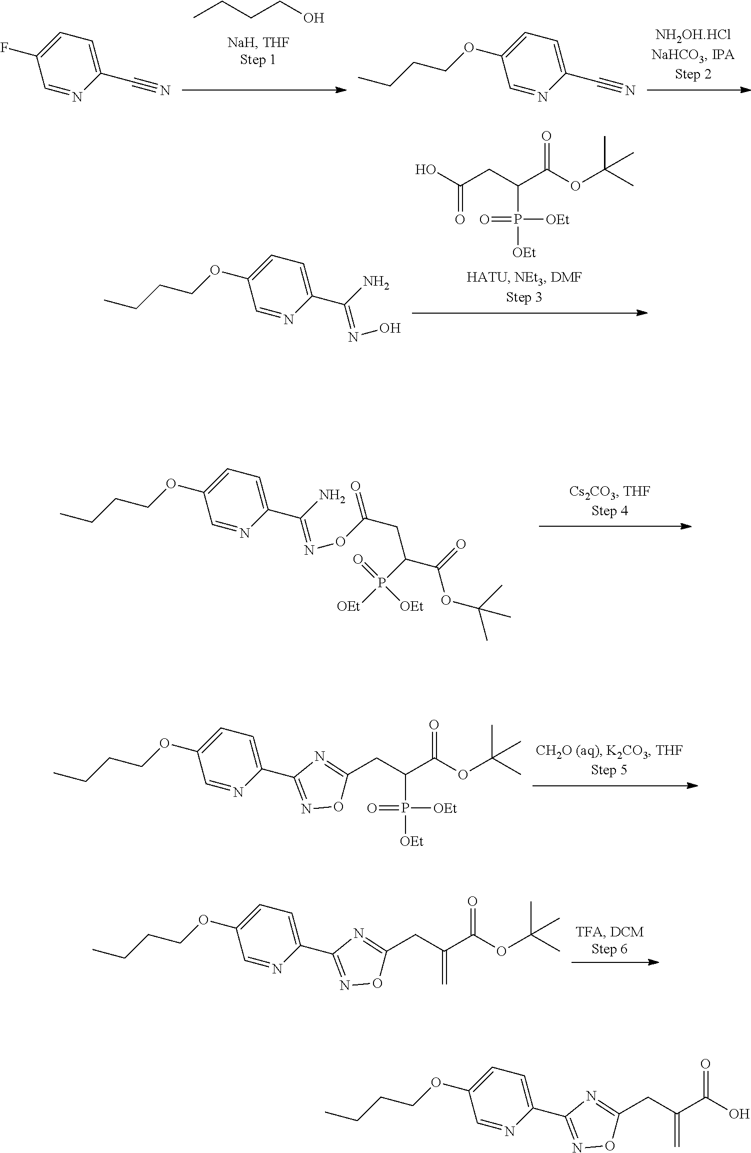

### Step 1

To a solution of butan-1-ol (6.68 g, 90.1 mmol) in THF (150 mL) was added sodium hydride (60 wt % dispersion in mineral oil, 3.96 g, 99.1 mmol) in portions at 0° C. The mixture was stirred at RT for 1 h, followed by addition of 5-fluoropicolinonitrile (11 g, 90.1 mmol) at 0° C. portionwise over 5 mins. The mixture was stirred at RT for 2 h then quenched with aq. sat. NH₄Cl (50 mL). The mixture was extracted with EtOAc (2×50 mL). The combined organic layers were washed with brine (50 mL), dried over Na₂SO₄, filtered and concentrated. The crude product was purified by chromatography on silica gel (0-18% MTBE/petroleum ether) to give 5-butoxypicolinonitrile (11 g, 62.4 mmol) as a colorless oil. LCMS (System 2, Method B) m/z 177.3 (M+H)⁺ (ES⁺).

### Step 2

To a mixture of hydroxylamine hydrochloride (1.18 g, 17 mmol) and sodium bicarbonate (1.46 g, 17 mmol) in IPA (30 mL) was stirred at RT for 30 mins and then 5-butoxypicolinonitrile (2.0 g, 11.3 mmol) was added. The resulting suspension was stirred at 60° C. for 18 h. The mixture was cooled to RT, filtered and concentrated to give (Z)-5-butoxy-N′-hydroxypicolinimidamide (2.30 g, 11 mmol) as a white solid. LCMS (System 2, Method C) m/z 210.4 (M+H)⁺ (ES⁺).

### Step 3

A mixture of 5-butoxy-N′-hydroxypicolinimidamide (2.0 g, 9.6 mmol), 4-tert-butoxy-3-(diethoxyphosphoryl)-4-oxobutanoic acid (Intermediate 1, 3 g, 9.6 mmol), HATU (5.44 g, 14.3 mmol) and triethylamine (2.9 g, 28.7 mmol) in N,N-dimethylformamide (30 mL) was stirred at RT for 1 h. The reaction was quenched with aq. sat. NH₄Cl (30 mL) and then extracted with EtOAc (2×30 mL). The combined organic layers were washed with water (2×20 mL) and brine (20 mL), dried over Na₂SO₄, filtered and concentrated to give tert-butyl (Z)-4-(((amino (5-butoxypyridin-2-yl)methylene) amino) oxy)-2-(diethoxyphosphoryl)-4-oxobutanoate (3.8 g, 7.6 mmol) as a light brown oil. The crude product was used in the next step directly. LCMS (System 2, Method C) m/z 502.4 (M+H)⁺ (ES⁺).

### Step 4

A mixture of tert-butyl (Z)-4-(((amino (5-butoxypyridin-2-yl)methylene) amino) oxy)-2-(diethoxyphosphoryl)-4-oxobutanoate (3.8 g, 7.6 mmol) and cesium carbonate (5.23 g, 16.1 mmol) in THF (30 mL) was stirred at 70° C. for 18 h. The reaction was filtered and concentrated. The crude product was purified by chromatography on silica gel (50-80% MTBE/petroleum ether) to give tert-butyl 3-(3-(5-butoxypyridin-2-yl)-1,2,4-oxadiazol-5-yl)-2-(diethoxyphosphoryl) propanoate (2.80 g, 5.8 mmol) as a light brown oil. LCMS (System 2, Method C) m/z 484.3 (M+H)⁺ (ES⁺).

### Step 5

To a mixture of tert-butyl 3-(3-(5-butoxypyridin-2-yl)-1,2,4-oxadiazol-5-yl)-2-(diethoxyphosphoryl) propanoate (1.4 g, 2.9 mmol) and potassium carbonate (800 mg, 5.8 mmol) in THF (10 mL) was added formaldehyde (37 wt % aqueous solution, 470 mg, 5.8 mmol) at 0° C., and the reaction was stirred at 0° C. for 1 h. The reaction was diluted with water (5 mL) and extracted with MTBE (2×10 mL) The combined organic layers were washed with brine (10 mL), dried over Na₂SO₄, filtered and concentrated. The crude product was purified by chromatography on silica gel (0-30% MTBE/petroleum ether) to give tert-butyl 2-((3-(5-butoxypyridin-2-yl)-1,2,4-oxadiazol-5-yl)methyl) acrylate (600 mg, 1.7 mmol) as a light brown oil. LCMS (System 2, Method C) m/z 360.3 (M+H)⁺ (ES⁺).

### Step 6

A solution of tert-butyl 2-((3-(5-butoxypyridin-2-yl)-1,2,4-oxadiazol-5-yl)methyl) acrylate (500 mg, 1.4 mmol) in TFA (1 mL) and DCM (3 mL) was stirred at RT for 1.5 h. The reaction was concentrated and the resulting residue was dissolved in DCM (3 mL). The pH was adjusted to 5-6 with aq. HCl (0.5 N) and the solvent was removed under reduced pressure. The crude product was purified by prep-HPLC(Column: Waters X-Bridge C18 OBD 10 μm 19×250 mm; Flow Rate: 20 mL/min; solvent system: MeCN/(0.05% TFA/water) gradient: MeCN: 55-95%; collection wavelength: 214 nm). The prep-HPLC fractions were concentrated at 30° C. under reduced pressure to remove MeCN, and the residue was lyophilized to give 2-((3-(5-butoxypyridin-2-yl)-1,2,4-oxadiazol-5-yl)methyl)acrylic acid (326 mg, 1.1 mmol) as a white solid. ¹H NMR (400 MHz, DMSO-d6) δ: 12.88 (br, 1H), 8.42 (d, J=2.8 Hz, 1H), 7.98 (d, J=8.8 Hz, 1H), 7.55 (dd, J=8.8, 3.2 Hz, 1H), 6.33 (s, 1H), 6.00 (s, 1H), 4.13 (d, J=6.4 Hz, 2H), 4.02 (s, 2H), 1.77-1.70 (m, 2H), 1.48-1.42 (m, 2H), 0.94 (t, J=7.2 Hz, 3H). LCMS (System 2, Method B) m/z 304.1 (M+H)⁺ (ES⁺).

### Example 3:2-((3-(5-butoxy-3-chloropyridin-2-yl)-1,2,4-oxadiazol-5-yl)methyl)acrylic acid

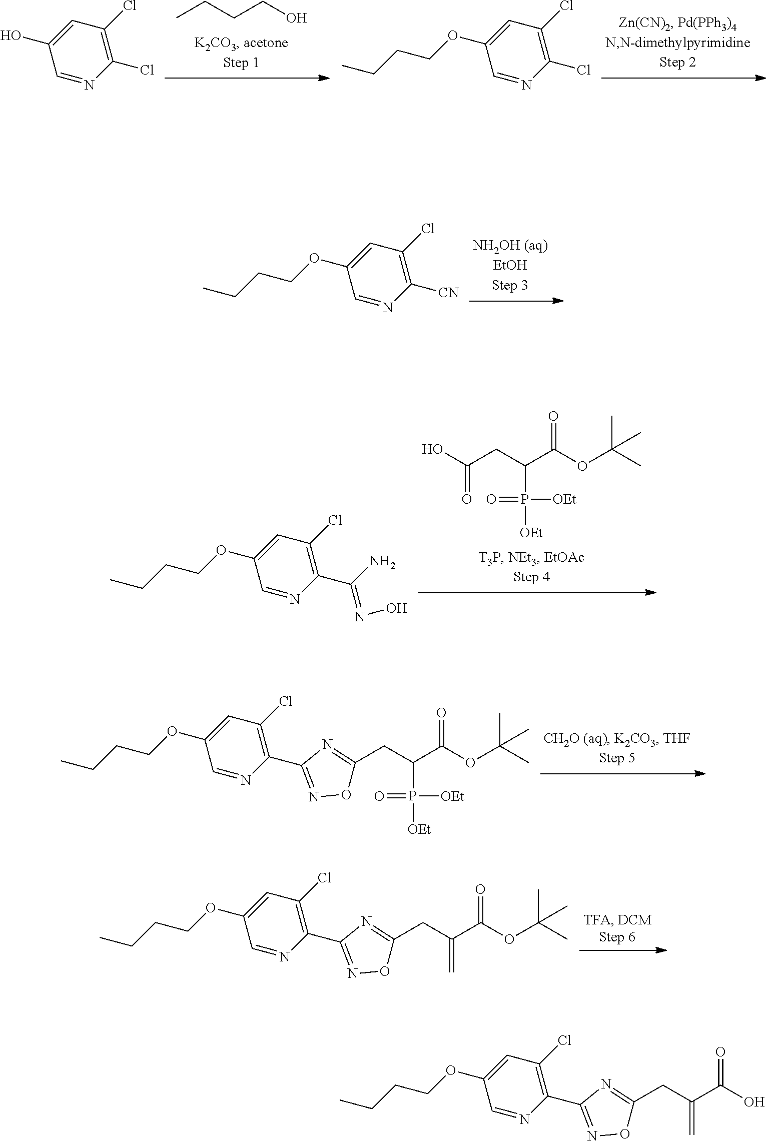

### Step 1

A mixture of 5,6-dichloropyridin-3-ol (2.2 g, 13.5 mmol), 1-bromobutane (5.51 g, 40.5 mmol) and potassium carbonate (2.42 g, 17.5 mmol) in acetone (60 mL) was stirred at 70° C. for 18 h. The reaction was filtered and concentrated. The crude product was purified by chromatography on silica gel (0-13% MTBE/petroleum ether) to give 5-butoxy-2,3-dichloropyridine (2.5 g, 11.3 mmol) as a colourless oil. LCMS (System 2, Method B) m/z 220.1 (M+H)⁺ (ES⁺).

### Step 2

A mixture of 5-butoxy-2,3-dichloropyridine (2.4 g, 10.95 mmol), Zn (CN) 2 (1.4 g, 12.1 mmol) and Pd(PPh₃)₄ (1.30 g, 1.1 mmol) in N,N-dimethylformamide (55 mL) was stirred at 100° C. for 18 h. The reaction was quenched with water (60 mL) and the mixture was extracted with EtOAc (2×80 mL). The combined organic layers were washed with brine (150 mL), dried over Na₂SO₄, filtered and concentrated. The crude product was purified by chromatography on silica gel (0-23% MTBE/petroleum ether) to give 5-butoxy-3-chloropicolinonitrile (1.50 g, 7.1 mmol) as a colourless oil. LCMS (System 2, Method C) m/z 211.4 (M+H)⁺ (ES⁺).

### Step 3

A mixture of 5-butoxy-3-chloropicolinonitrile (1.50 g, 7.1 mmol) and hydroxylamine (50% aqueous solution, 1.41 g, 21.4 mmol) in EtOH (35 mL) was stirred at 90° C. for 18 h. The mixture was cooled to RT and concentrated to give (Z)-5-butoxy-3-chloro-N′-hydroxypicolinimidamide (1.70 g, 6.7 mmol) as a light yellow oil. LCMS (System 2, Method C) m/z 244.4 (M+H)⁺ (ES⁺).

### Step 4

A mixture of (Z)-5-butoxy-3-chloro-N′-hydroxypicolinimidamide (1.70 g, 6.7 mmol), 4-tert-butoxy-3-(diethoxyphosphoryl)-4-oxobutanoic acid (Intermediate 1, 2.16 g, 6.7 mmol) and triethylamine (2.12 g, 21 mmol) in EtOAc (20 mL) was added 2,4,6-tripropyl-1,3,5,2,4,6-trioxatriphosphinane 2,4,6-trioxide (50 wt % in EtOAc, 8.89 g, 14 mmol) below 25° C. The reaction was stirred at 80° C. for 16 h. The reaction was quenched with aq. HCl (0.5 N, 30 mL) and the mixture was extracted with EtOAc (2×40 mL). The combined organic layers were washed with brine (60 mL), dried over Na₂SO₄, filtered and concentrated. The crude product was purified by chromatography on silica gel (0-56% MTBE/petroleum ether) to give tert-butyl 3-(3-(5-butoxy-3-chloropyridin-2-yl)-1,2,4-oxadiazol-5-yl)-2-(diethoxyphosphoryl) propanoate (1.50 g, 2.9 mmol) as a light yellow oil. LCMS (System 2, Method C) m/z 518.2 (M+H)⁺ (ES⁺).

### Step 5

A mixture of tert-butyl 3-(3-(5-butoxy-3-chloropyridin-2-yl)-1,2,4-oxadiazol-5-yl)-2-(diethoxyphosphoryl) propanoate (1.50 g, 2.9 mmol) and potassium carbonate (520 mg, 3.8 mmol) in THF (15 mL) was added formaldehyde (37 wt % aqueous solution, 0.71 mL, 8.7 mmol). The reaction was stirred at RT for 5 hours. The reaction was diluted with water (20 mL) and the layers separated. The aqueous layer was further extracted with MTBE (3×20 mL). The combined organic layers were washed with brine (60 mL), dried over Na₂SO₄, filtered and concentrated. The crude product was purified by chromatography on silica gel (0-15% MTBE/petroleum ether) to give tert-butyl 2-((3-(5-butoxy-3-chloropyridin-2-yl)-1,2,4-oxadiazol-5-yl)methyl) acrylate (900 mg, 2.3 mmol) as a light yellow oil. LCMS (System 2, Method C) m/z 394.3 (M+H)⁺ (ES⁺).

### Step 6

A solution of tert-butyl 2-((3-(5-butoxy-3-chloropyridin-2-yl)-1,2,4-oxadiazol-5-yl)methyl) acrylate (900 mg, 2.3 mmol) in TFA (3 mL) and DCM (3 mL) was stirred at RT for 1.5 h. The mixture was concentrated and the crude residue was purified by prep-HPLC(Column: Waters X-Bridge C18 OBD 10 μm 19×250 mm; Flow Rate: 20 mL/min; solvent system: MeCN/(0.05% TFA/water) gradient: MeCN: 65-95%; collection wavelength: 214 nm). The prep-HPLC fractions were concentrated at 30° C. under reduced pressure to remove MeCN, and the residue was lyophilized to give 2-((3-(5-butoxy-3-chloropyridin-2-yl)-1,2,4-oxadiazol-5-yl)methyl)acrylic acid (605 mg, 1.8 mmol) as a white solid. ¹H NMR (400 MHz, DMSO-d6) δ: 12.89 (br, 1H), 8.42 (d, J=2.8 Hz, 1H), 7.80 (d, J=2.4 Hz, 1H), 6.32 (d, J=0.4 Hz, 1H), 6.00 (d, J=1.2 Hz, 1H), 4.17 (t, J=6.4 Hz, 2H), 4.05 (s, 2H), 1.77-1.70 (m, 2H), 1.45 (q, J=7.6 Hz, 2H), 0.95 (t, J=7.2 Hz, 3H). LCMS (System 2, Method B) m/z 338.2 [M+H]⁺ (ES⁺)

### Example 4: 2-((3-(3-chloro-5-(4-fluorophenoxy)pyridin-2-yl)-1,2,4-oxadiazol-5-yl)methyl)acrylic acid

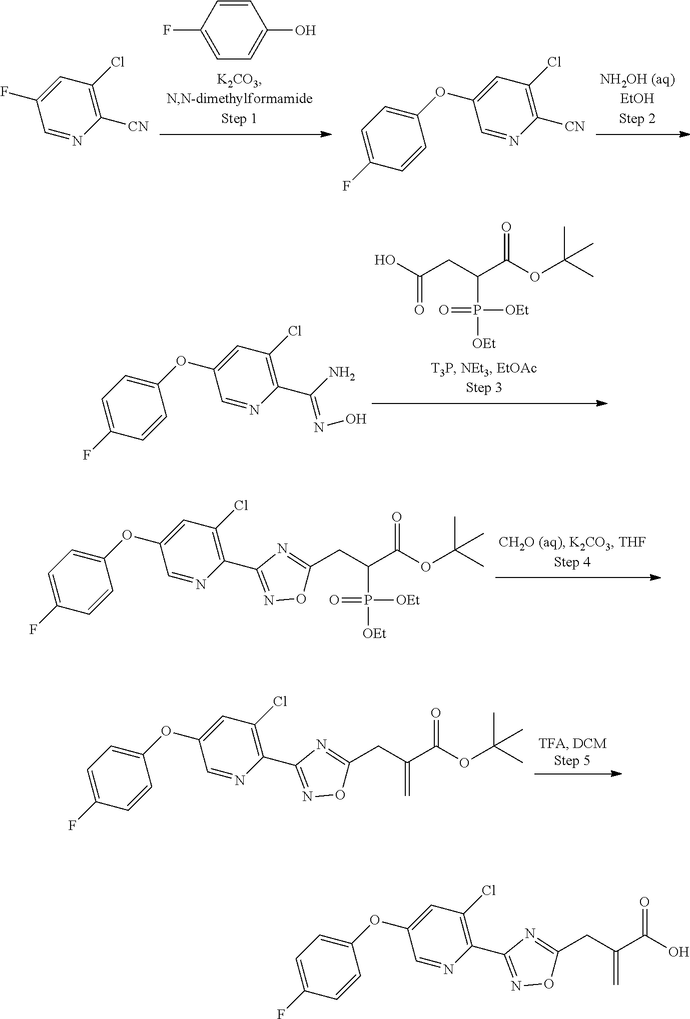

### Step 1

A mixture of 3-chloro-5-fluoropicolinonitrile (350 mg, 2.2 mmol), 4-fluorophenol (376 mg, 3.3 mmol) and potassium carbonate (401 mg, 2.9 mmol) in DMSO (10 mL) was stirred at RT for 16 h. The mixture was quenched with water (15 mL) and extracted with EtOAc (3×15 mL). The combined organic layers were washed with brine (30 mL), dried over Na₂SO₄, filtered and concentrated. The crude product was purified by chromatography on silica gel (0-20% MTBE/petroleum ether) to give 3-chloro-5-(4-fluorophenoxy) picolinonitrile (550 mg, 2.1 mmol, 95% purity) as a colourless oil. LCMS (System 2, Method C) m/z 249.2 (M+H)⁺ (ES⁺).

### Step 2

A mixture of 3-chloro-5-(4-fluorophenoxy) picolinonitrile (550 mg, 2.1 mmol, 95% purity) and hydroxylamine (50% aqueous solution, 70 mg, 6.3 mmol) in EtOH (10 mL) was stirred at 60° C. for 2 h. The reaction was cooled to RT and concentrated to give (Z)-3-chloro-5-(4-fluorophenoxy)-N′-hydroxypicolinimidamide (560 mg, 1.9 mmol, 98% purity) as a white solid. LCMS (System 2, Method C) m/z 282.2 (M+H)⁺ (ES⁺).

### Step 3

A mixture of (Z)-3-chloro-5-(4-fluorophenoxy)-N′-hydroxypicolinimidamide (560 mg, 1.9 mmol, 98% purity), 4-tert-butoxy-3-(diethoxyphosphoryl)-4-oxobutanoic acid (Intermediate 1, 604 mg, 1.9 mmol) and triethylamine (591 g, 5.8 mmol) in EtOAc (10 mL) was added 2,4,6-tripropyl-1,3,5,2,4,6-trioxatriphosphinane 2,4,6-trioxide (50 wt % in EtOAc, 2.48 g, 3.9 mmol) at 5° C. The reaction was stirred at 80° C. for 16 h. The reaction was quenched with aq. HCl (0.5 N, 20 mL) and extracted with EtOAc (3×15 mL). The combined organic layers were washed with brine (40 mL), dried over Na₂SO₄, filtered and concentrated. The crude product was purified by chromatography on silica gel (0-100% MTBE/petroleum ether) to give tert-butyl 3-(3-(3-chloro-5-(4-fluorophenoxy)pyridin-2-yl)-1,2,4-oxadiazol-5-yl)-2-(diethoxyphosphoryl) propanoate (600 mg, 1.1 mmol, 98% purity) as a colourless oil. LCMS (System 2, Method C) m/z 556.2 (M+H)⁺ (ES⁺).

### Step 4

A mixture of tert-butyl 3-(3-(3-chloro-5-(4-fluorophenoxy)pyridin-2-yl)-1,2,4-oxadiazol-5-yl)-2-(diethoxyphosphoryl) propanoate (600 mg, 1.05 mmol, 98% purity) and potassium carbonate (190 mg, 1.4 mmol) in THF (5 mL) was added formaldehyde (37 wt % aqueous solution, 258 mg, 3.2 mmol), and the reaction mixture was stirred at 25° C. for 3 h. The reaction mixture was diluted with water (10 mL) and extracted with MTBE (3×15 mL). The combined organic layers were washed with brine (40 mL), dried over Na₂SO₄, filtered and concentrated. The crude product was purified by chromatography on silica gel (0-50% MTBE/petroleum ether) to give tert-butyl 2-((3-(3-chloro-5-(4-fluorophenoxy)pyridin-2-yl)-1,2,4-oxadiazol-5-yl)methyl) acrylate (340 mg, 0.8 mmol, 99% purity) as a colourless oil. LCMS (System 2, Method C) m/z 432.2 (M+H)⁺ (ES⁺).

### Step 5

A solution of tert-butyl 2-((3-(3-chloro-5-(4-fluorophenoxy)pyridin-2-yl)-1,2,4-oxadiazol-5-yl)methyl) acrylate (340 mg, 0.8 mmol, 99% purity) in TFA (2 mL) and DCM (4 mL) was stirred at RT for 1.5 h. The reaction was concentrated and the residue was purified by prep-HPLC(Column: Waters X-Bridge C18 OBD 10 μm 19×250 mm; Flow Rate: 20 mL/min; solvent system: MeCN/(0.05% TFA/water) gradient: MeCN: 65-95%; collection wavelength: 214 nm). The prep-HPLC fractions were concentrated at 35° C. under reduced pressure to remove MeCN, and the residue was lyophilized to give 2-((3-(3-chloro-5-(4-fluorophenoxy)pyridin-2-yl)-1,2,4-oxadiazol-5-yl)methyl)acrylic acid (219 mg, 0.6 mmol, 99% purity) as a white solid. ¹H NMR (400 MHz, DMSO-d6) δ 12.89 (br, 1H), 8.48 (d, J=2.4 Hz, 1H), 7.72 (d, J=2.4 Hz, 1H), 7.33 (t, J=4.8 Hz, 4H), 6.32 (d, J=0.8 Hz, 1H), 6.00 (d, J=1.2 Hz, 1H), 4.07 (s, 2H). LCMS (System 2, Method B) m/z 376.0 [M+H]⁺ (ES⁺).

### Biological Example 1—THP-1 AlphaLISA IL-1β

Measuring Inhibitory Effects on IL-1β Cytokine Output from THP-1s

The cytokine inhibition profiles of compounds of formula (I) were determined in a differentiated THP-1 cell assay. All assays were performed in RPMI-1640 growth medium (Gibco), supplemented with 10% fetal bovine serum (FBS; Gibco), 1% penicillin-streptomycin and 1% sodium pyruvate unless specified otherwise. The IL-1β cytokine inhibition assay was run in a background of differentiated THP-1 cells as described below. All reagents described were from Sigma-Aldrich unless specified otherwise. Compounds were prepared as 10 mM DMSO stocks.

### Assay Procedure

THP-1 cells were expanded as a suspension up to 80% confluence in appropriate growth medium. Cells were harvested, suspended, and treated with an appropriate concentration of phorbol 12-myristate 13-acetate (PMA) over a 72 hr period (37° C./5% CO₂).

Following 72 hrs of THP-1 cell incubation, cellular medium was removed and replaced with fresh growth media containing 1% of FBS. Working concentrations of compounds were prepared separately in 10% FBS treated growth medium and pre-incubated with the cells for 30 minutes (37° C./5% CO₂). Following the 30 minute compound pre-incubation, THP-1s were treated with an appropriate concentration of LPS and the cells were subsequently incubated for a 24 hr period (37° C./5% CO₂). An appropriate final concentration of Nigericin was then dispensed into the THP-1 plates and incubated for 1 hour (37° C./5% CO₂) before THP-1 supernatants were harvested and collected in separate polypropylene 96-well holding plates.

Reagents from an IL-1β and IL-6 commercial kit (Perkin Elmer) were prepared and run according to the manufacturer's instructions. Subsequently, fluorescence signal detection in a microplate reader was measured (EnVision® Multilabel Reader, Perkin Elmer).

Percentage inhibition was calculated per cytokine by normalising the sample data to the high and low controls used within each plate (+/−LPS respectively). Percentage inhibition was then plotted against compound concentration and the 50% inhibitory concentration (IC₅₀) was determined from the resultant concentration-response curve.

The compounds of formula (I) were tested and the results are shown in Table 1 below. 4-Octyl itaconate, 2-(2-chlorobenzyl)acrylic acid (Cocco et al., 2017) and monomethylfumarate were included as comparator compounds.

TABLE 1
THP-1 cell IL-1β and IL-6 IC₅₀ values (μM)

| Compound | IL-1β (IC₅₀) | IL-6 (IC₅₀) |
|---|---|---|
| 4-octyl itaconate | 37.8 | 19.1 |
| 2-(2-chlorobenzyl)acrylic | >100 | NT^{§} |
| acid | | |
| monomethyl fumarate | >100 | >100 |
| Example 1 | >100 | 10.3 |
| Example 2 | >100 | 21.4 |
| Example 3 | 2.09 | 6.71 |
| Example 4 | 2.12 | 2.52 |

^{§}Not tested in this assay

These results reveal that certain compounds of the present invention are expected to have anti-inflammatory activity as shown by their IC₅₀ values for inhibition of IL-1β and/or IL-6 release in this assay. All example compounds tested exhibited improved IL-1β and/or IL-6 lowering properties compared with monomethyl fumarate. All examples exhibited improved or similar IL-6 lowering properties (IC₅₀ values) compared to 4-octyl itaconate. Certain examples exhibited improved IL-1β lowering properties (IC₅₀ values) compared to 4-octyl itaconate. Certain examples exhibited improved IL-13 lowering properties (IC₅₀ values) compared to 2-(2-chlorobenzyl)acrylic acid. Examples 1 and 2 were not effective in the IL-1β assay.

### Biological Example 2—NRF2+/−GSH Activation Assay

### Measuring Compound Activation Effects on the Anti-Inflammatory Transcription Factor NRF2 in DiscoverX PathHunter NRF2 Translocation Kit

Potency and efficacy of compounds of formula (I) against the target of interest to activate NRF2 (nuclear factor erythroid 2-related factor 2) were determined using the PathHunter NRF2 translocation kit (DiscoverX). The NRF2 translocation assay was run using an engineered recombinant cell line, utilising enzyme fragment complementation to determine activation of the Keap1-NRF2 protein complex and subsequent translocation of NRF2 into the nucleus. Enzyme activity was quantified using a chemiluminescent substrate consumed following the formation of a functional enzyme upon PK-tagged NRF2 translocation into the nucleus.

The assay was run under both +/−GSH (glutathione) conditions to determine the sensitivity of the compounds' NRF2-activating abilities to attenuation by GSH.

Additionally, a defined concentration of DMF was used as the ‘High’ control to normalise test compound activation responses to.

### Assay Procedure

U2OS PathHunter express cells were thawed from frozen prior to plating. Following plating, U2OS cells were incubated for 24 hrs (37° C./5% CO₂) in commercial kit provided cell medium.

Following 24 hrs of U2OS incubation, cells were directly treated with an appropriate final concentration of compound, for −GSH conditions or, for +GSH conditions, an intermediate plate containing 6× working concentrations of compound stocks was prepared in a 6 mM working concentration of GSH solution (solubilised in sterile PBS). Following a 30 minute compound-GSH pre-incubation (37° C./5% CO₂) for +GSH treatment, plated U2OS cells were incubated with an appropriate final concentration of compound and GSH.

Following compound (+/−GSH) treatment, the U2OS plates were incubated for a further 6 hours (37° C./5% CO₂) before detection reagent from the PathHunter NRF2 commercial kit was prepared and added to test plates according to the manufacturer's instructions. Subsequently, the luminescence signal detection was measured in a microplate reader (PHERAstar®, BMG Labtech).

Percentage activation was calculated by normalising the sample data to the high and low controls used within each plate (+/−DMF). Percentage activation/response was then plotted against compound concentration and the 50% activation concentration (EC₅₀) was determined from the plotted concentration-response curve.

A number of compounds of formula (I) were tested, and the results are shown in Table 2 below. 4-Octyl itaconate and 2-(2-chlorobenzyl)acrylic acid were included as comparator compounds.

TABLE 2
NRF2 activation

| | −GSH | | +GSH | |
|---|---|---|---|---|
| Compound | EC₅₀ (μM) | Eₘₐₓ (%) | EC₅₀ (μM) | Eₘₐₓ (%) |
| 4-octyl itaconate | 22.1 | 165 | 28.9 | 148 |
| 2-(2-chlorobenzyl)acrylic | >100 | 2 | >100 | 9 |
| acid | | | | |
| Example 1 | 20.8 | 192.5 | 19.5 | 130 |
| Example 3 | 22.7 | 172.5 | 11.4 | 78.58 |
| 4-octyl itaconate | 22.1 | 165 | 28.9 | 148 |
| 2-(2-chlorobenzyl)acrylic | >100 | 2 | >100 | 9 |
| acid | | | | |
| Example 4 | 7.07 | 208.56 | 15.5 | 145 |

These results reveal that compounds of the present invention are expected to have anti-inflammatory activity as shown by their EC₅₀ and/or Eₘₐₓ values for NRF2 activation in this assay. All compounds shown in Table 2 exhibited lower EC₅₀ and higher Eₘₐₓ values in both-GSH and +GSH compared to 2-(2-chlorobenzyl)acrylic acid. Certain compounds shown in Table 2 exhibited lower EC₅₀ and/or high Eₘₐₓ values in one or both of −GSH and +GSH compared to 4-octyl itaconate.

### Biological Example 3—Hepatocyte Stability Assay

Defrosted cryo-preserved hepatocytes (viability >70%) were used to determine the metabolic stability of a compound via calculation of intrinsic clearance (Cₗᵢₙₜ; a measure of the removal of a compound from the liver in the absence of blood flow and cell binding). Clearance data are particularly important for in vitro work as they can be used in combination with in vivo data to predict the half-life and oral bioavailability of a drug.

The metabolic stability in hepatocytes assay involves a time-dependent reaction using both positive and negative controls. The cells were pre-incubated at 37° C. then spiked with test compound (and positive control); samples were taken at pre-determined time intervals and were analysed to monitor the change in concentration of the initial drug compound over 60 minutes. A buffer incubation reaction (with no hepatocytes present) acted as a negative control and two cocktail solutions, containing compounds with known high and low clearance values (verapamil/7-hydroxycoumarin and propranolol/diltiazem), acted as positive controls.
1. The assay was run with a cell concentration of 0.5×10⁶ cells/mL in Leibovitz buffer.
2. All compounds and controls were run in duplicate.
3. Compound concentration was 10 μM.
4. All compounds and controls were incubated with both cells and buffer to show turnover was due to hepatic metabolism.
5. All wells on the incubation plate had 326.7 μL of either cells or buffer added.
6. Prior to assay, cell and buffer-only incubation plates were preincubated for 10 mins at 37° C.
7. The assay was initiated by adding compounds, 3.3 μL of 1 mM in 10% DMSO-90% Buffer; final DMSO concentration was 0.1%.
8. Samples were taken at regular timepoints (0, 5, 10, 20, 40, 60 min) until 60 mins.
9. Sample volume was 40 μL and was added to 160 μL of crash solvent (acetonitrile with internal standard) and stored on ice.
10. At the end of the assay, the crash plates were centrifuged at 3500 rpm for 20 mins at 4° C.
11. 80 μL of clear supernatant was removed and mixed with 80 μL of deionised water before being analysed by LC-MS/MS.

Raw LC-MS/MS data was exported to, and analysed in, Microsoft Excel for determination of intrinsic clearance. The percentage remaining of a compound was monitored using the peak area of the initial concentration as 100%. Intrinsic clearance and half-life values were calculated using a graph of the natural log of percentage remaining versus the time of reaction in minutes. Half-life (min) and intrinsic clearance (Clᵢₙₜ in μL min⁻¹ 10⁻⁶ cells) values were calculated using the gradient of the graph (the elimination rate constant, k) and Equations 1 and 2.

$${t}_{\frac{1}{2}} = \frac{ln 2}{k} \qquad \{ Equation 1 \}$$

$${Cl}_{int} = ( \frac{ln 2}{{t}_{\frac{1}{2}}} ) \times ( \frac{350}{0.175} ) \qquad \{ Equation 2 \}$$

An example of a compound of formula (I) was tested, and the results are shown in Table 3 below. 4-Octyl itaconate and 2-(2-chlorobenzyl)acrylic acid (Cocco et al., 2017) were included as comparator compounds.

TABLE 3
Hepatocyte stability

| Compound | Species | Clᵢₙₜ (μL min⁻¹ 10⁻⁶ cells) | T½ (min) |
|---|---|---|---|
| 4-octyl itaconate | Mouse | 351 | 4 |
| | Human | 401 | 4 |
| 2-(2- | Mouse | 95 | 11 |
| chlorobenzyl)acrylic | Human | 21 | 59 |
| acid | | | |
| Example 3 | Mouse | 133 | 9.27 |
| | Human | 7.18 | 181 |

These results reveal that compounds of the invention are expected to have acceptable metabolic stabilities, as shown by their intrinsic clearance (Clᵢₙₜ) and half-life (T_{1/2}) values in this assay. Example 3 was more stable, i.e., exhibited lower intrinsic clearance (Clᵢₙₜ) and had a longer half-life (T_{1/2} values) in mouse and/or human cells compared to 4-octyl itaconate and 2-(2-chlorobenzyl)acrylic acid.

### REFERENCES

The following publications cited in this specification are herein incorporated by reference in their entirety.
Ackermann et al. Proc. Soc. Exp. Bio. Med. 1949, 72 (1), 1-9.
Andersen J. L. et al. Nat. Commun. 2018, 9, 4344.
Angiari S. and O'Neill L. A. Cell Res. 2018, 28, 613-615.
Bagavant G. et al. Indian J. Pharm. Sci. 1994, 56, 80-85.
Bambouskova M. et al. Nature 2018, 556, 501-504.
Blewett M. M. et al. Sci. Sign. 2016, 9 (445), rs10; 6.
Brennan M. S. et al. PLOS One 2015, 10, e0120254.
Brück J. et al. Exp. Dermatol. 2018, 27, 611-624.
Cocco M. et al. J. Med. Chem. 2014, 57, 10366-10382.
Cocco M. et al. J. Med. Chem. 2017, 60, 3656-3671.
Cordes T. et al. J. Biol. Chem. 2016, 291, 14274-14284.
Cordes T. et al. Mol. Metab. 2020, 32, 122-135.
Daly R. et al. medRxiv 2019, 19001594; doi: https://doi.org/10.1101/19001594.
Daniels B. P. et al. Immunity 2019, 50 (1), 64-76.e4.
Dibbert S. et al. Arch. Dermatol. Res. 2013, 305, 447-451.
ElAzzouny M. et al. J. Biol. Chem. 2017, 292, 4766-4769.
Gillard G. O. et al. J. Neuroimmunol. 2015, 283, 74-85.
Gu L. et al. Immunol. Cell Biol. 2020, 98 (3), 229-241.
Hanke T. et al. Pharmacol. Therapeut. 2016, 157, 163-187.
Hunt T. et al. Consortium of Multiple Sclerosis Centers 2015 Annual Meeting, 27-30 May 2015,
Indianapolis, IN, USA: Poster DX37.
Kobayashi E. H. et al. Nat. Commun. 2016, 7, 11624.
Kornberg M. D. et al. Science 2018, 360, 449-453.
Kulkarni R. A. et al. Nat. Chem. Biol. 2019, 15, 391-400.
Lampropoulou V. et al. Cell Metab. 2016, 24, 158-166.
Lehmann J. C. U. et al. J. Invest. Dermatol. 2007, 127, 835-845.
Liao S.-T. et al. Nat. Commun. 2019, 10 (1), 5091.
Liu H. et al. Cell Commun. Signal. 2018, 16, 81.
McGuire V. A. et al. Sci. Rep. 2016, 6, 31159.
Michelucci A. et al. Proc. Natl. Acad. Sci. USA 2013, 110, 7820-7825.
Mills E. A. et al. Front. Neurol. 2018, 9, 5.
Mills E. L. et al. Cell 2016, 167, 457-470.
Mills E. L. et al. Nature 2018, 556, 113-117.
Mrowietz U. et al. Trends Pharmacol. Sci. 2018, 39, 1-12.
Müller S. et al. J. Dermatol. Sci. 2017, 87, 246-251.
Murphy M. P. and O'Neill L. A. J. Cell 2018, 174, 780-784.
O'Neill L. A. J. and Artyomov M. N. Nat. Rev. Immunol. 2019 273-281.
Olagnier D. et al. Nat. Commun. 2018, 9, 3506.
Schmidt T. J. et al. Bioorg. Med. Chem. 2007, 15, 333-342.
Shan Q. et al. Biochem. Biophys. Res. Commun. 2019, 517, 538-544.
Straub R. H. and Schradin C. Evol. Med. Public Health 2016, 1, 37-51S.
Straub R. H. and Cutolo M. Rheumatology 2016, 55 (Suppl. 2), ii6-ii14.
Sun X. et al., FASEB J. 2019, 33, 12929-12940.
Tang C. et al. Cell Physiol. Biochem. 2018, 51, 979-990.
Tang C. et al. Biochem. Biophys. Res. Commun. 2019, 508, 921-927.
Tang H. et al. Biochem. Biophys. Res. Commun. 2008, 375, 562-565.
Tian et al. Eur. J. Pharmacol. 2020, 873, 172989.
van der Reest J. et al. Nat. Commun. 2018, 9, 1581.
von Glehn F. et al. Mult. Scler. Relat. Disord. 2018, 23, 46-50.
Yi F. et al. Hepatology 2020, 873, 172989.
Yu X.-H. et al. Immunol. Cell Biol. 2019, 97, 134-141.
Zhang D. et al. Int. Immunopharmacol. 2019, 77, 105924.
Zhang S. et al. Bioorg. Med. Chem. 2012, 20, 6073-6079.
Zhao C. et al. Microb. Pathogen. 2019, 133, 103541.
Zhao G. et al. Biochem. Biophys. Res. Commun. 2014, 448, 303-307.

### Miscellaneous

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

1. A compound of formula (I): 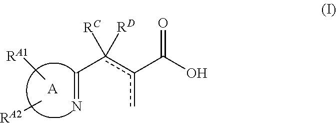
wherein, 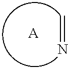
represents a 5 membered heteroaryl ring, which in addition to the C═N shown contains one or more further heteroatoms independently selected from N, O and S;
or 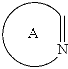
represents a 6 membered heteroaryl ring, which in addition to the C═N shown optionally contains one or more further N atoms;
R^{A1} is —(CH₂)₀₋₆-heteroaryl or O-heteroaryl;
wherein R^{A1} is optionally substituted by one or more substituents selected from the group consisting of halo, C₁₋₆ alkyl, C₁₋₆ haloalkyl, hydroxy, cyano, OG¹, S(O)₀₋₂G¹, SFs, (CH₂)₀₋₃C₃₋₇ cycloalkyl and 5-7-membered heterocyclyl wherein said C₃₋₇ cycloalkyl and said 5-7-membered heterocyclyl are optionally substituted by one or more groups selected from halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms;
or R^{A1} is optionally substituted by one phenyl ring which is optionally substituted by C₁₋₂ haloalkyl, C₁₋₂ haloalkoxy or one or more halo atoms;
wherein G¹ is C₁₋₆ alkyl, C₃₋₇ cycloalkyl, C₁₋₆ haloalkyl, or (CH₂)₀₋₁phenyl, wherein G¹ is optionally substituted by one or more substituents selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR¹R², OG² and S(O)₀₋₂G²;
wherein G² is C₁₋₆ alkyl, C₃₋₇ cycloalkyl, C₁₋₆ haloalkyl, or phenyl which is optionally substituted by one or more substituents selected from the group consisting of halo, C₁₋₂ alkyl, C₁₋₂ haloalkyl, hydroxy, cyano, nitro, C₁₋₂ alkoxy and C₁₋₂ haloalkoxy; and
wherein R¹ and R² are independently H or C₁₋₂ alkyl or, taken together, R¹ and R² may combine to form a 5-7-membered heterocyclic ring;
or R^{A2} is absent; and
R^{C} and R^{D} are each independently H, C₁₋₂ alkyl, hydroxy, fluoro or C₁₋₂ alkoxy; or R^{C} and
R^{D} may join to form a C₃₋₅ cycloalkyl ring;
wherein 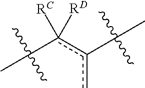
in the compound of formula (I) represents: 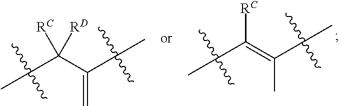
and wherein
the total number of carbon atoms in groups R^{A1} and R^{A2} taken together including their optional substituents is 6-14;
or a pharmaceutically acceptable salt and/or solvate thereof.

2. The compound or a pharmaceutically acceptable salt and/or solvate thereof according to claim 1 which is a compound of formula (I).

3. The compound or a pharmaceutically acceptable salt and/or solvate thereof according to claim 1 which is a pharmaceutically acceptable salt of a compound of formula (I).

4. The compound or a pharmaceutically acceptable salt and/or solvate thereof according to
claim 1 wherein 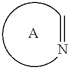
is 1,2,4-oxadiazole.

5. The compound or a pharmaceutically acceptable salt and/or solvate thereof according to claim 1 wherein R^{A1} is —(CH₂)₀₋₆-heteroaryl such as —(CH₂)₀-heteroaryl e.g. pyridyl.

6. The compound or a pharmaceutically acceptable salt and/or solvate thereof according of claim 1 wherein R^{A1} is substituted by halo and OG¹.

7. The compound or a pharmaceutically acceptable salt and/or solvate thereof according to claim 1 wherein G¹ is C₁₋₆ alkyl e.g., n-butyl, or phenyl substituted by halo.

8. The compound according to claim 1, selected from the group consisting of:
2-((3-(6-butoxypyridin-3-yl)-1,2,4-oxadiazol-5-yl)methyl)acrylic acid;
2-((3-(5-butoxypyridin-2-yl)-1,2,4-oxadiazol-5-yl)methyl)acrylic acid;
2-((3-(3-2-((3-(5-butoxy-3-chloropyridin-2-yl)-1,2,4-oxadiazol-5-yl)methyl)acrylic acid; and
chloro-5-(4-fluorophenoxy)pyridin-2-yl)-1,2,4-oxadiazol-5-yl)methyl)acrylic acid;
or a pharmaceutically acceptable salt and/or solvate of any one thereof.

9. A pharmaceutical composition comprising a compound or a pharmaceutically acceptable salt and/or solvate thereof according to claim 1 and one or more pharmaceutically acceptable diluents or carriers.

10. (canceled)

11. (canceled)

12. (canceled)

13. A method of treating or preventing an inflammatory disease or a disease associated with an undesirable immune response, which comprises administering to a patient in need of such treatment an effective amount of a compound or pharmaceutically acceptable salt and/or solvate thereof according to claim 1.

14. The method according to claim 13 wherein the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenia purpura, thrombotic thrombocytopenia purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-) encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C(HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

15. The method according to claim 14, wherein the inflammatory disease or disease associated with an undesirable immune response is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

16. The method according to claim 14, wherein the inflammatory disease or disease associated with an undesirable immune response is selected from the group consisting of rheumatoid arthritis; psoriatic arthritis; systemic lupus erythematosus; multiple sclerosis; psoriasis; Crohn's disease; ulcerative colitis; juvenile idiopathic arthritis; uveitis; spondyloarthrapathies; ankylosing spondylitis; temporal arteritis; polymyalgia rheumatica; erosive osteoarthritis of the hands; Lupus nephritis; Parkinson's disease; inflammatory bowel disease; celiac disease; dermatomyositis; hidradenitis suppurativa; Sjogren's syndrome; giant cell arteritis (temporal arteritis); systemic juvenile idiopathic arthritis (Still's disease); familial Mediterranean fever (FMF); tumour necrosis factor (TNF) receptor-associated periodic fever syndrome (TRAPS); hyperimmunoglobulinaemia D with periodic fever syndrome (HIDS); cryopyrin-associated periodic syndromes (CAPS); Aicardi-Goutières syndrome; and spondyloenchondrodysplasia.

17. The method according to claim 13, for use in combination with a further therapeutic agent, such as a corticosteroid (glucocorticoid), retinoid (e.g. acitretin, isotretinoin, tazarotene), anthralin, vitamin D analogue (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, a thiopurine (e.g. azathioprine, 6-mercaptopurine), methotrexate, an anti-TNFα agents (e.g. infliximab, etanercept, adalimumab, certolizumab, golimumab or a biosimilar), phosphodiesterase-4 (PDE4) inhibition (e.g. apremilast, crisaborole), anti-IL-17 agent (e.g. brodalumab, ixekizumab, secukinumab), anti-IL12/IL-23 agent (e.g. ustekinumab, briakinumab), anti-IL-23 agent (e.g. guselkumab, tildrakizumab), JAK (Janus Kinase) inhibitor (e.g. tofacitinib, ruxolitinib, baricitinib, filgotinib, upadacitinib), plasma exchange, intravenous immune globulin (IVIG), cyclophosphamide, anti-CD20 B cell depleting agent (e.g. rituximab, ocrelizumab, ofatumumab, obinutuzumab), anthracycline analogue (e.g. mitoxantrone), cladribine, sphingosine 1-phosphate receptor modulator or sphingosine analogue (e.g. fingolimod, siponimod, ozanimod, etrasimod), interferon beta preparation (including interferon beta 1b/1a), glatiramer, anti-CD3 therapy (e.g. OKT3), anti-CD52 targeting agent (e.g. alemtuzumab), leflunomide, teriflunomide, gold compound, laquinimod, potassium channel blocker (e.g. dalfampridine/4-aminopyridine), mycophenolic acid, mycophenolate mofetil, purine analogue (e.g. pentostatin), mTOR (mechanistic target of rapamycin) pathway inhibitor (e.g. sirolimus, everolimus), anti-thymocyte globulin (ATG), IL-2 receptor (CD25) inhibitor (e.g. basiliximab, daclizumab), anti-IL-6 receptor or anti-IL-6 agent (e.g. tocilizumab, siltuximab), Bruton's tyrosine kinase (BTK) inhibitor (e.g. ibrutinib), tyrosine kinase inhibitor (e.g. imatinib), ursodeoxycholic acid, hydroxychloroquine, chloroquine, B cell activating factor (BAFF, also known as BLyS, B lymphocyte stimulator) inhibitor (e.g. belimumab, blisibimod), other B cell targeted therapy including a fusion protein targeting both APRIL (A PRoliferation-Inducing Ligand) and BLyS (e.g. atacicept), PI3K inhibitor including pan-inhibitor or one targeting the p110δ and/or p110γ containing isoforms (e.g. idelalisib, copanlisib, duvelisib), an interferon α receptor inhibitor (e.g. anifrolumab, sifalimumab), T cell co-stimulation blocker (e.g. abatacept, belatacept), thalidomide and its derivatives (e.g. lenalidomide), dapsone, clofazimine, a leukotriene antagonist (e.g. montelukast), theophylline, anti-IgE therapy (e.g. omalizumab), an anti-IL-5 agent (e.g. mepolizumab, reslizumab), a long-acting muscarinic agent (e.g. tiotropium, aclidinium, umeclidinium), a PDE4 inhibitor (e.g. roflumilast), riluzole, a free radical scavenger (e.g. edaravone), a proteasome inhibitor (e.g. bortezomib), a complement cascade inhibitor including one directed against C5 (e.g. eculizumab), immunoadsor, antithymocyte globulin, 5-aminosalicylates and their derivatives (e.g. sulfasalazine, balsalazide, mesalamine), an anti-integrin agent including one targeting α4β1 and/or α4β7 integrins (e.g. natalizumab, vedolizumab), an anti-CD11-α agent (e.g. efalizumab), a non-steroidal anti-inflammatory drug (NSAID) including a salicylate (e.g. aspirin), a propionic acid (e.g. ibuprofen, naproxen), an acetic acid (e.g. indomethacin, diclofenac, etodolac), an oxicam (e.g. meloxicam) a fenamate (e.g. mefenamic acid), a selective or relatively selective COX-2 inhibitor (e.g. celecoxib, etroxicoxib, valdecoxib and etodolac, meloxicam, nabumetone), colchicine, an IL-4 receptor inhibitor (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, a specific antibiotic with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, a cystic fibrosis transmembrane conductance regulator (CFTR) modulator, a VEGF (vascular endothelial growth factor) inhibitor (e.g. bevacizumab, ranibizumab, pegaptanib, aflibercept), pirfenidone or mizoribine.

18. A process for preparing the compound of formula (I): 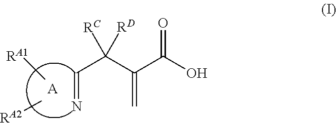
or a salt, such as a pharmaceutically acceptable salt thereof which comprises hydrolysing the ester moiety in a compound of formula (II): 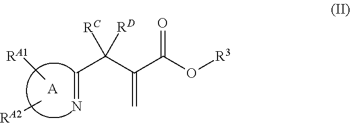
or a salt thereof,
wherein 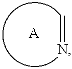
R^{A1}, R^{A2}, R^{C} and R^{D} are as defined in any one of claims 1 to 7, and R³ is C₁₋₄ alkyl optionally substituted with halo.

19. A compound which is selected from the group consisting of:
a compound of formula (II): 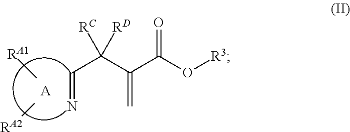
and
a compound of formula (III): 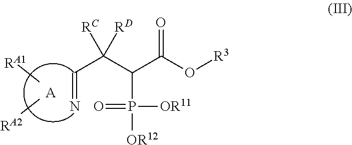
or salt thereof,
wherein in any of the above compounds, 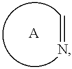
R^{A1}, R^{A2}, R^{C} and R^{D} are as defined in any one of claims 1 to 7, and R¹¹, R¹² and R³ are independently C₁₋₄ alkyl optionally substituted with halo.
